(19) United States
(12) Patent Application Publication
(10) Pub. No.: US 2022/0000871 A1
(43) Pub. Date: Jan. 6, 2022

(54) METHODS AND COMPOSITIONS FOR TREATING NEURODEGENERATIVE DISEASES USING MODULATORS OF PHOSPHOGLYCERATE KINASE 1 (PGK1) ACTIVITY

(71) Applicants: UNIVERSITY OF IOWA RESEARCH FOUNDATION, Iowa City, IA (US); CAPITAL MEDICAL UNIVERSITY, Beijing (CN)

(72) Inventors: Lei LIU, Beijing (CN); Michael J. WELSH, Riverside, IA (US)

(21) Appl. No.: 17/290,149

(22) PCT Filed: Oct. 28, 2019
(86) PCT No.: PCT/US2019/058378

§ 371 Date: Apr. 29, 2021

(51) Int. Cl. A61K 31/517; A61K 31/4725; A61P 25/28; A61P 25/16

(52) U.S. Cl. CPC A61K 31/517; A61P 25/16; A61P 25/28; A61K 31/4725

(57) ABSTRACT

Disclosed are methods and compositions for treating and/or preventing neurodegenerative diseases or disorders in a subject in need thereof. The methods may include administering to the subject a pharmaceutical composition comprising an effective amount of a therapeutic agent that binds and/or activates phosphoglycerate kinase 1 (PGK1). Neurodegenerative diseases or disorders treated by the disclosed methods may include Parkinson's disease (PD), Alzheimer's disease (AD), Huntington's disease (HD), amyotrophic lateral sclerosis (ALS), and/or Lewy body dementia.

### CROSS-REFERENCE TO RELATED PATENT APPLICATIONS

The present application is a national stage filing under 35 U.S.C. § 371 of International Application No. PCT/US2019/058378, filed Oct. 28, 2019, which claims the benefit of priority to international application PCT/CN2018/112402, filed on Oct. 29, 2018, which application lapsed prior to publication.

### BACKGROUND

The field of the invention relates to methods and compositions for treating and/or preventing a neurodegenerative disease or disorder or symptoms thereof by administering a therapeutic agent that activates phosphoglycerate kinase 1 (PGK1) activity to a subject in need thereof. Neurodegenerative diseases and disorders treated by the disclosed methods may include, but are not limited to Parkinson's disease, Alzheimer's disease, Huntington's disease, amyotrophic lateral sclerosis, and Lewy body dementia.

Neurodegenerative diseases such as Parkinson's disease, Alzheimer's disease, Huntington's disease, amyotrophic lateral sclerosis, Lewy body dementia, and diseases that exhibit protein aggregates and premature apoptosis exact enormous human, medical and economic burdens. Although in some cases limited symptomatic relief can be provided, there are no treatments that halt or slow progression of the neurodegeneration.

A key pathogenic factor in Parkinson's disease is impaired energy metabolism and generation of ATP. Impaired energy metabolism is also a shared feature in Alzheimer's disease, Huntington disease, and otherr neurodegenerative diseases. Arun, S., Liu, L., and Donmez, G. (2016). Mitochondrial biology and neurological diseases. Curr. Neuropharmacol. 14, 143-154

Earlier studies revealed that terazosin binds and stimulates phosphoglycerate kinase 1 (PGK1), thereby increasing glycolysis and ATP levels in cells. Increasing PGK1 activity and raising ATP levels may be beneficial, even when the ATP level in the cell is not reduced because it could further reduce protein aggregate formation. For example, in cells, raising ATP levels decreases aggregates. Therefore, to test the hypothesis that terazosin would increase ATP levels and prevent neurodegeneration, we used Parkinson's disease as a model of a common neurodegenerative disease. We discovered that terazosin reverses energy deficits in models of Parkinson's disease in mice, rats, flies, and induced pluripotent stem cells from patients with Parkinson's disease. We learned that terazosin prevents or slows neuronal loss. It also increases tyrosine hydroxylase and dopamine levels in surviving neurons and partially restores motor function, even when begun after the onset of neurodegeneration.

We asked if terazosin would have a beneficial effect and alter the course of disease in humans with Parkinson's disease. Therefore, we examined the Parkinson's Progession Markers Initiative database and discovered that terazosin use was associated with slower decline in motor function in patients with Parkinson's disease. We also examined the Truven Health Analytics MarketScan Database and found that use of terazosin and two closely related drugs that also enhance PGK1 activity (doxazosin and alfuzosin) decreased Parkinson's disease symptoms and complications.

Impaired energy metabolism and protein aggregation also are key features of many other neurodegenerative diseases, including Alzheimer's disease, Huntington's disease, amyotrophic lateral sclerosis, Lewy body dementia, and others. The increased levels of ATP produced when terazosin enhances PGK1 activity are likely key to terazosin's effect on neurodegeneration. Previous studies have shown that ATP has properties of a hydrotrope. At physiological concentrations, ATP both prevents formation of and dissolves previously formed protein aggregates. As ATP concentrations increase, solubilization increases. Thus, by elevating ATP levels, terazosin may facilitate solubilization of aggregates, including α-synuclein, and prevent cell dysfunction and death in many neurodegenerative diseases. In addition, ATP generated from Pgk1 may also enhance the chaperone activity of Hsp90, an ATPase known to associate with Pgk1. Upon activation, Hsp90 is known to promote multistress resistance.

In addition to enhancing PGK1 activity, terazosin is also an antagonist of the α1-adrenergic receptor. Terazosin is an FDA approved drug that is used clinically to treat benign prostatic hypertrophy and hypertension because it inhibits the αl-adrenergic receptor and thereby relaxes smooth muscle. Thus, terazosin has two targets, the αl-adrenergic receptor and PGK1. However, for treating Parkinson's disease and other neurodegenerative diseases, eliminating the al-adrenergic receptor antagonist activity would be advantageous. A limitation of terazosin and related agents that enhance PGK1 activity, but also inhibit al-adrenergic receptors is that they reduce autonomic activity and can cause hypotension and orthostatic hypotension. For example, terazosin could exacerbate the autonomic dysfunction and orthostatic hypotension observed in patients with Parkinson's disease. Moreover, orthostatic hypotension is a common problem in older people and worsens with advancing age; increasing age is a well-known risk factor for Parkinson's disease, Alzheimer's disease, and other neurodegenerative diseases.

Here, we propose methods and compositions for treating neurodegenerative diseases and disorders by administering a therapeutic agent that activates phosphoglycerate kinase 1 (PGK1) activity. In some embodiments, the therapeutic agent binds and activates phosphoglycerate kinase 1 (PGK1) selectively with minimal off-target effects.

### SUMMARY

Disclosed are methods and compositions for treating and/or preventing a neurodegenerative diseases or disorders or symptoms thereof in a subject in need thereof. The methods may include administering to the subject a pharmaceutical composition comprising an effective amount of a therapeutic agent that binds and/or activates phosphoglycerate kinase 1 (PGK1).

The disclosed methods and compositions may be utilized to treat and/or prevent neurodegenerative diseases or disorders or symptoms thereof. Suitable neurodegenerative diseases or disorders that may be treated by the disclosed methods and compositions may include, but are not limited to Parkinson's disease (PD), Alzheimer's disease (AD), Huntington's disease (HD), amyotrophic lateral sclerosis (ALS), and Lewy body dementia. Symptoms of neurodegenerative disease or disorders may include but are not limited to sleep disturbances, depression, and weakness. More severe symptoms may include dementia, neuropsychiatric disease, and movement disorders.

In some embodiments, the disclosed methods include administering to a subject having AD, HD, ALS, and/or Lewy body dementia, a therapeutic agent that binds and/or activates PGK1 selected from terazosin, prazosin, doxozosin, alfuzosin, trimazosin, and abanoquil.

In some embodiments, the therapeutic agent does not bind to the α₁-adrenergic receptor (α₁AR) and/or does not function as a ligand for the α₁AR as an agonist or antagonist. In some embodiments of the disclosed methods and compositions, the therapeutic agent may be selected from compounds characterized as having a substituted isoquinoline core or a substituted quinazoline core.

### BRIEF DESCRIPTION OF THE FIGURES

FIG. 1. TZ enhances glycolysis in the mouse brain. In all figures, data points are from individual mice, rats, or groups of flies. Bars and whiskers indicate mean±SEM. Blue indicates controls and red indicates TZ treatment. A) Schematic of ATP production by glycolysis and oxidative phosphorylation. B) Schematic time course for experiments in panels C-G. Eight week-old C57bl/6 mice were given MPTP (20 mg/kg i.p.) or vehicle 4 times at 2 hr intervals. Then, TZ (10 μg/kg) or vehicle were injected i.p. once a day for one week. Assays were at day 7. C-E) Pyruvate levels (C), citrate synthase (CS) activity (D), and ATP levels (E) measured in mouse striatum. In panel E, TZ doses are indicated. N=6. Statistical comparison is vs. 0 TZ. F,G) Pyruvate (F) and ATP (G) levels in mouse striatal region. In the figures, *p<0.05, **p<0.01, ***p<0.001. For panels C and D the statistical test was Mann-Whitney, for panels E, a Kruskal-Wallis with a Dunn's test, and for panels F and G, a Kruskal-Wallis with a Dwass-Steele-Critchlow-Fligner.

TZ improves dopamine neuron and motor function in MPTP-treated mice. A) Schematic for experiments in panels B-K. C57BL/6 mice (8 week-old) received 4 i.p. injections of MPTP (20 mg/kg at 2 hr intervals) or vehicle on day 0. Mice were then injected with TZ (10 μg/kg) or vehicle (0.9% saline) once a day for one week and assays were performed on day 7. Other mice began receiving daily TZ or vehicle injections beginning on day 7 and assays were performed on day 14. N=6. B-D) Example of western blots with TH and β-actin (protein loading control) in striatum and SNc at days 7 and 14 (B). Quantification of TH protein normalized to control (C,D). N=6. E-G) Example of immunostaining of TH in SNc and striatum (E, scale bars, 500 μm SNc, 1 mm striatum). Quantification of TH-positive neurons in SNc (F) and TH intensity in the striatum (G). N=6. H,I) Dopamine (DA) content in striatum and SnC. N=6. J) Percentage of TH-positive neurons that were positive for TUNEL staining. N=6. K) Behavioral response of mice in rotarod test. Data are duration that mice remained on an accelerated rolling rod normalized to mice at day 0. N=8. Data are examples and mean±SEM. Blue indicates control and red indicates TZ treatment. Statistical analysis at day 7 and day 14 was Mann-Whitney. In the figure, *p<0.05, **p<0.01.

FIG. 3. TZ slows neurodegeneration, increases dopamine, and improves motor performance in 6-OHDA-treated rats. A) Schematic for experiments in panels B-G. 6-OHDA (20 μg) was injected into right striatum of rats on day 0. TZ (70 μg/kg) or saline were injected (i.p.) daily for 2 weeks, beginning 2, 3, 4, or 5 weeks after 6-OHDA injection. Assays were at 0 and 2-7 weeks. B) Percentage of SNc cells that were TUNEL-positive. N=6. C) Quantification of TH protein assessed by immunoblot in the striatum normalized to control. N=6. D,E) Percentage of SNc cells positive for TH immunostaining (D) and intensity of TH immunostaining in striatum (E) 7 weeks after 6-OHDA injection; TZ treatment was weeks 5-7. N=6. F) Dopamine content in right striatum relative to left (control) striatum. N=6. G) Results of cylinder test. 6-OHDA was injected into right striatum impairing use of left paw. Assay was 7 weeks after 6-OHDA injection; TZ treatment was week 5-7. N=4 for control group and 10 for the two 6-OHDA groups. In panels C, D, E, and data points are from individual rats. Bars and whiskers indicate mean±SEM. Blue indicates controls and red indicates TZ treatment. In the figures, *p<0.05, **p<0.01, ***p<0.001. For panels B and F, statistical test was Mann-Whitney, for panels C, D, and E, a Kruskal-Wallis with Dwass-Steele-Critchlow-Fligner, and for panel a Friedman with Dunn's.

FIG. 4. TZ enhances Pgk activity to attenuate rotenone-impaired motor performance. A) Schematic for experiments in panels B-F. Flies received rotenone (125 or 250 μM in food) with TZ (1 μM) or vehicle for 7 or 14 days. B) Relative ATP content in brains of w¹¹¹⁸ flies receiving 250 μM rotenone ±TZ for 14 days. N=6 with 200 fly heads for each treatment in each trial. C) Climbing behavior of flies after 250 μM rotenone with TZ (1 μM) or vehicle for 7 days. Data are percentage of flies that climbed up a tube (see Methods). N=3 with 200 flies tested for each treatment in each trial. D) Knockdown of Pgk in offspring of actin-Gal4 crossed with UAS-Pgk RNA″ flies. Offspring of actin-Gal4 crossed with y1 v1; P [CaryP] attP2 were used as a genetic background matched control. N=3 with RNA collected from 30 fly heads for each sample. E) Pgk was knocked down in TH neurons by crossing UAS-Pgk RNAi flies with flies carrying TH neuron-specific promoter (TH-Gal4) to produce TH>Pgk RNAi flies. Rotenone (250 μM) and TZ were administered as indicated for 7 days. Climbing behavior was measured on day 7. N=8 with 200 flies tested for each treatment in each trial. F) Pgk (UAS-Pgk) overexpression was driven by a dopaminergic neuron promoter (TH-Gal4), pan-neuronal promoter (Appl-Gal4), pan-cell promoter (Actin-Gal4), and muscle-specific promoter (Mhc-Gal4). Rotenone (250 μM) was administered for 7 days. Climbing behavior was measured on day 7. N=3, with 200 flies tested for each treatment in each trial. Data points are from individual groups of flies. Bars and whiskers indicate mean±SEM. Blue indicates controls and red indicates TZ treatment. In the figures, *p<0.05, **p<0.01, ***p<0.001. For panel B, the statistical test was a Kruskal-Wallis with Dwass-Steele-Critchlow-Fligner, for panel C and E, a 1-way ANOVA with Tukey, for panel D, a paired t-test, and for panel F, an unpaired t-test.

FIG. 5. TZ improves TH levels and motor performance in genetic models of PD. A-E) Wild-type (w¹¹¹⁸) and PINK1⁵ flies received TZ or vehicle for 10 days beginning on the first day after eclosion. Day 10 assays included: (A) Example of wing posture defect and percentage of w¹¹¹⁸ and PINK1⁵ flies with wing posture defects. N=6, with 80 flies for each treatment in each trial. (B,C) Example of TH western blot (B) and quantification of TH (C). N=5, with 40 fly heads for each treatment in each trial. (D) ATP content in brains (relative to w¹¹¹⁸). N=3, with 200 fly heads for each treatment in each trial. (E) Climbing behavior. N=3, with 100 flies for each treatment in each trial. F) Climbing behavior of LRRK^{ex1} male flies. N=6, with 100 flies for each treatment in each trial. G-K) TZ delivery to mThyl-hSNCA transgenic mice. (G) Schematic for experiments in panels H-K. (H) Example of western blot of α-synuclein in striatum and SNc. (I,J) Quantification of α-synuclein in striatum and SNc. N=5. (K) Duration that mice remained on an accelerating rotarod. N=5. Data are from individual groups of flies (A-F) and individual mice (I-K). Bars and whiskers indicate mean±SEM. Blue indicates controls and red indicates TZ treatment. In the figures, *p<0.05, **p<0.01, ***p<0.001. For panel D, the statistical analysis was 1-way ANOVA with Tukey, and for all other panels, a Kruskal-Wallis with a Dwass-Steele-Critchlow-Fligner.

FIG. 6. TZ increases ATP content and decreases α-synuclein accumulation in iPSC-derived dopamine neurons from PD patients. A) iPSC-derived dopamine neurons from two PD patients (Subjects 12 and 13) carrying LRRK2^{G2019S} mutations and a healthy control (Subject 11). 30-day old dopamine (DA) neurons were plated and began receiving TZ (10 μM) 1 or 3 days later. They were studied 24 hours after adding TZ. We observed no difference between the two start days and therefore combined the data. Representative immunofluorescence images of α-synuclein (SNCA, green), TH (red), and DAPI (nuclei, blue). B) Percentage of TH-positive neurons with cytoplasmic accumulation of α-synuclein. N=12. C) ATP content in control and LRRK2^{G2019S} iPSC-derived dopamine neurons. N=12. Bars and whiskers indicate mean±SEM. Blue indicates controls and red indicates TZ treatment. In the figures, *p<0.05, **p<0.01, ***p<0.001. Statistical analysis was a Mann-Whitney.

FIG. 7. TZ and related drugs slow progression of motor defects for patients with PD enrolled in the PPMI database. MDS-UPDRS Part 3 (motor) scores of PD patients in the PPMI database. Patients were taking TZ/DZ/AZ (blue, N=13), tamsulosin (green, N=24), or neither (red, N=269). Data are scores at entry into PPMI through ˜1 year and include all measures between those times. All patients taking these drugs were males prescribed TZ/DZ/AZ or tamsulosin without breaks for benign prostatic hyperplasia or undefined urological problems. Lines are plotted from linear mixed effect regression analyses. By maximum likelihood estimation, TZ/DZ/AZ differed from controls (P=0.012).

FIG. 8. TZ and related drugs reduce symptoms as assessed by diagnostic codes for patients with PD in the Truven/IBM Watson clinical database. Data are from the Truven Health Marketscan Commercial Claims and Encounters and Medicare Supplemental databases between 2011 and 2016. Patients had a diagnosis of PD and were prescribed TZ/DZ/AZ or tamsulosin for at least 1 year. We assessed relative risks for 79 previously identified PD-related diagnostic codes. A) Relative risk for 79 PD-related diagnostic codes for patients taking TZ/DZ/AZ vs. tamsulosin. Yellow indicates a statistically significant difference in risk between TZ/DZ/AZ and tamsulosin (P<0.05) determined by a generalized linear model with a quasi-Poisson distribution. B) Relative risk for the categories of PD-related diagnostic codes for patients taking TZ/DZ/AZ vs. tamsulosin. Data are means and 95% confidence intervals.

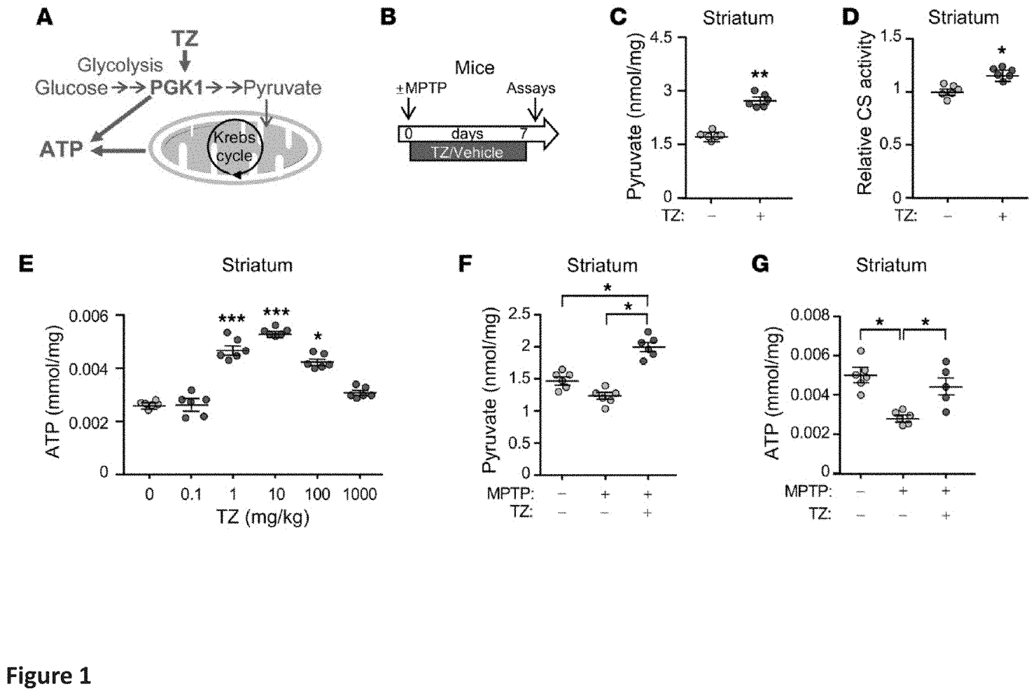

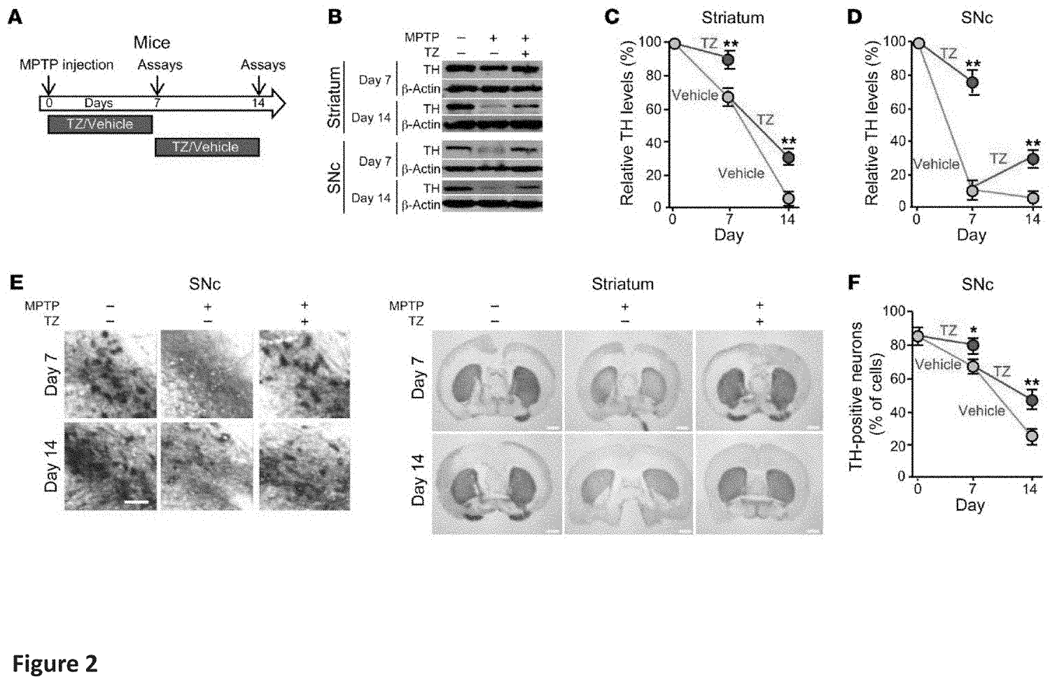

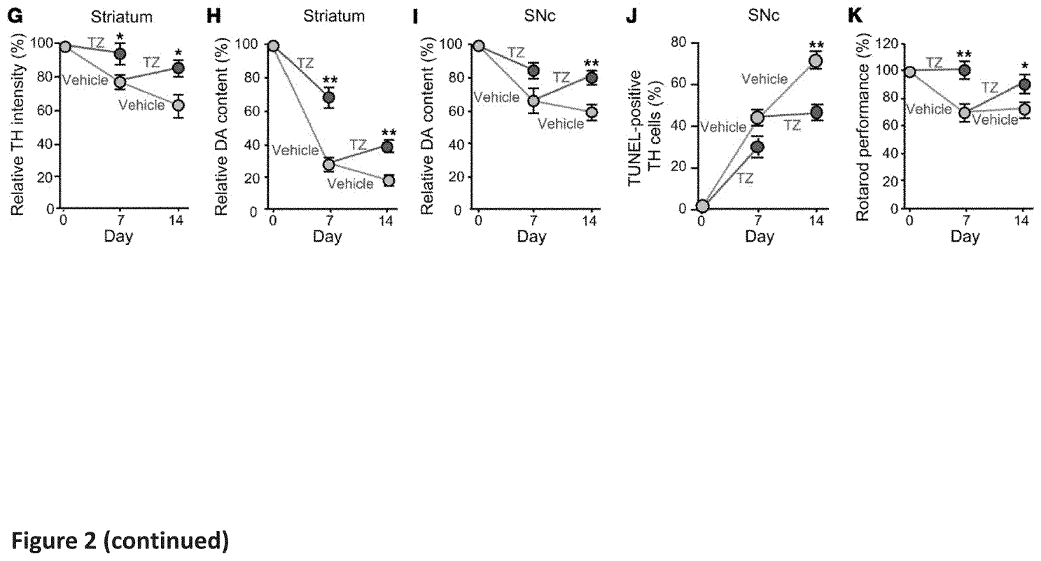

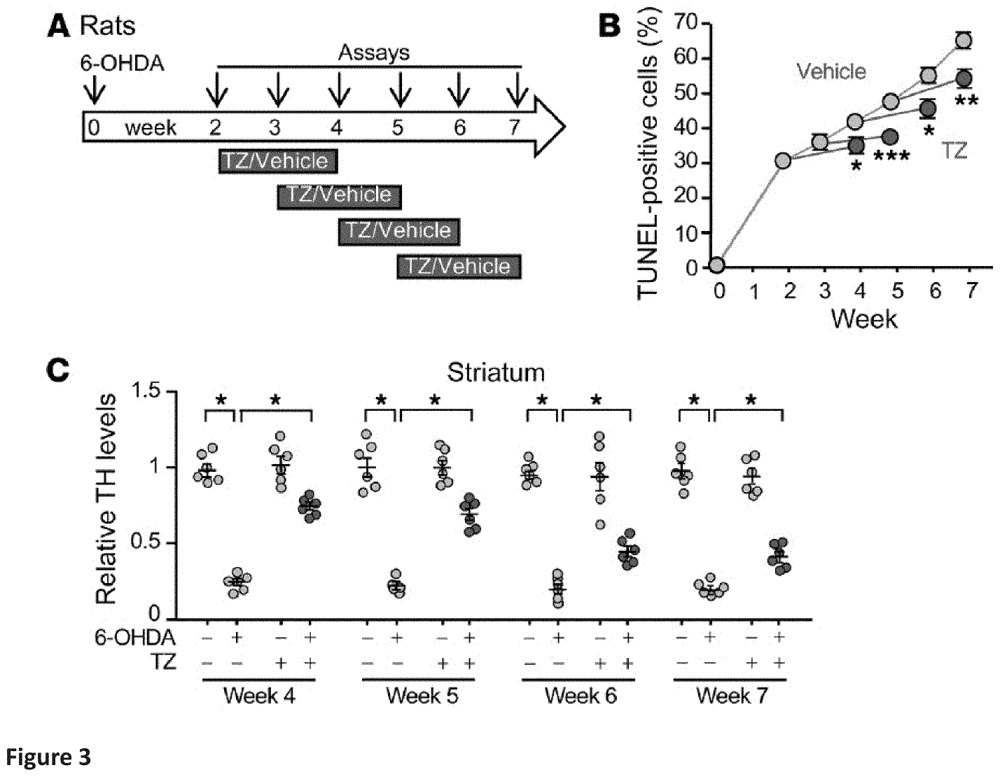

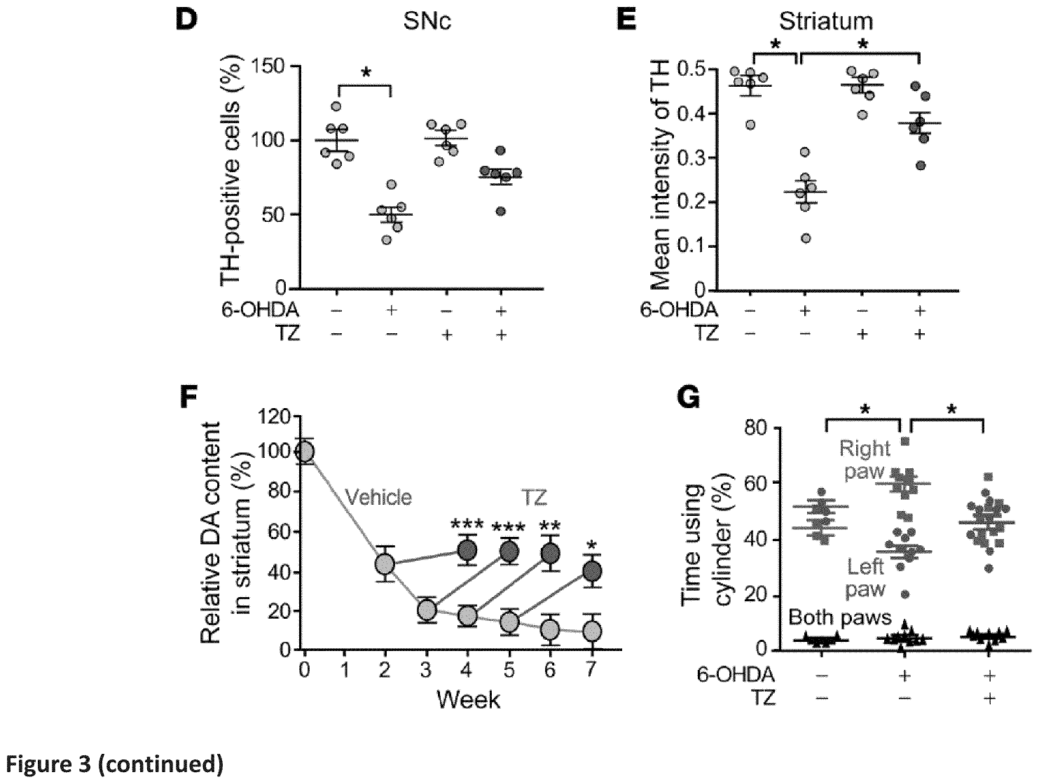

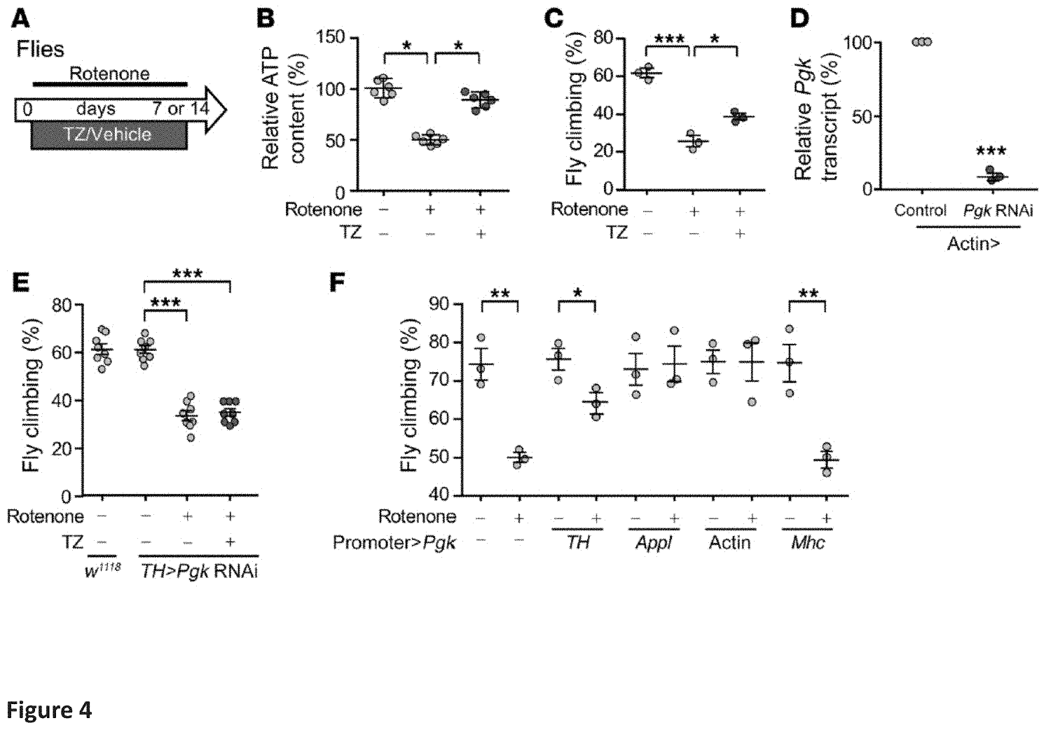

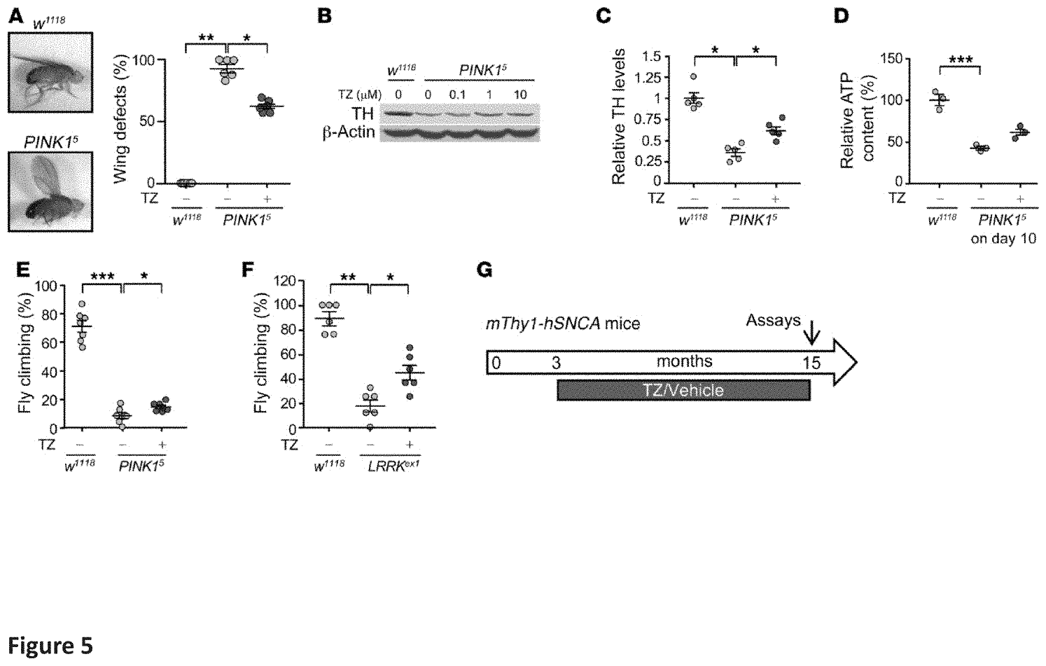

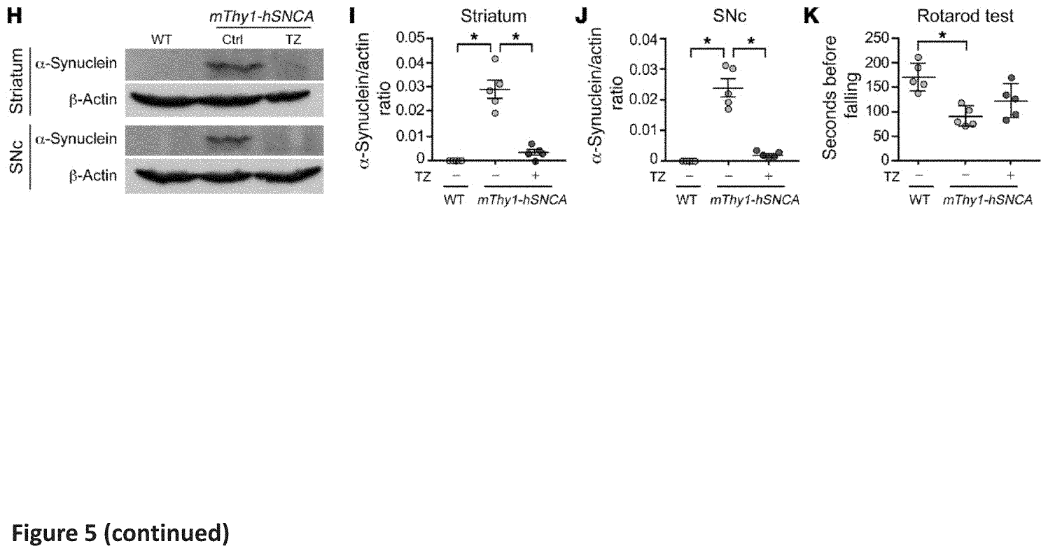

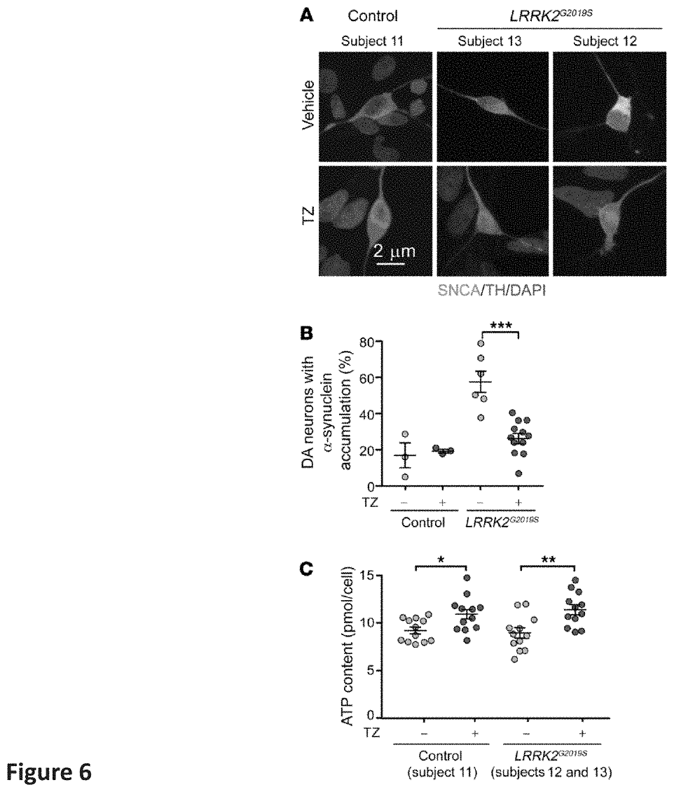

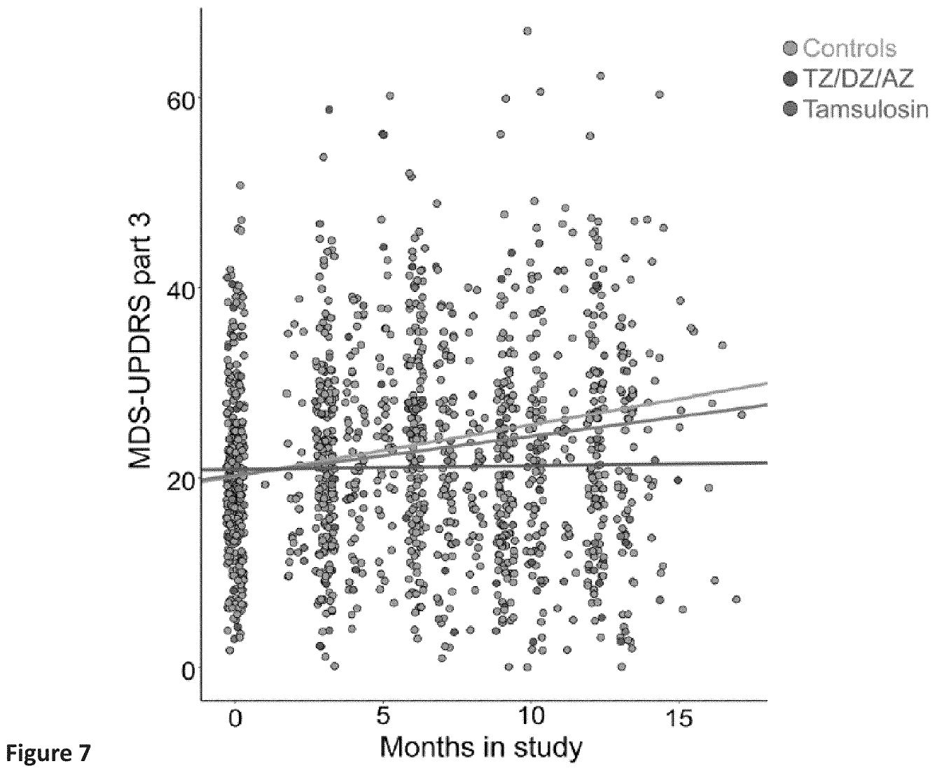

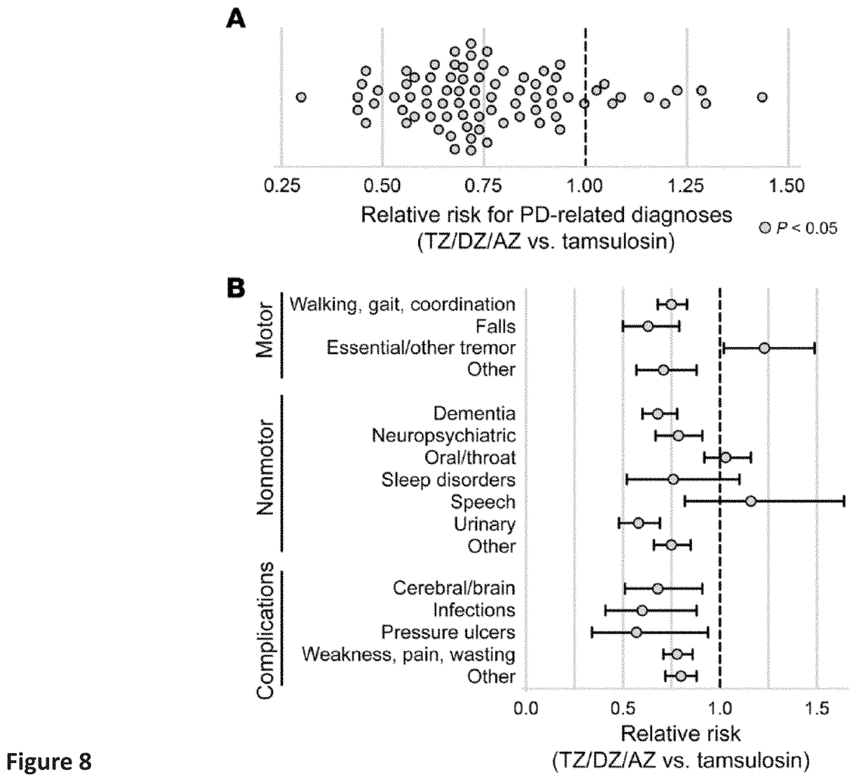

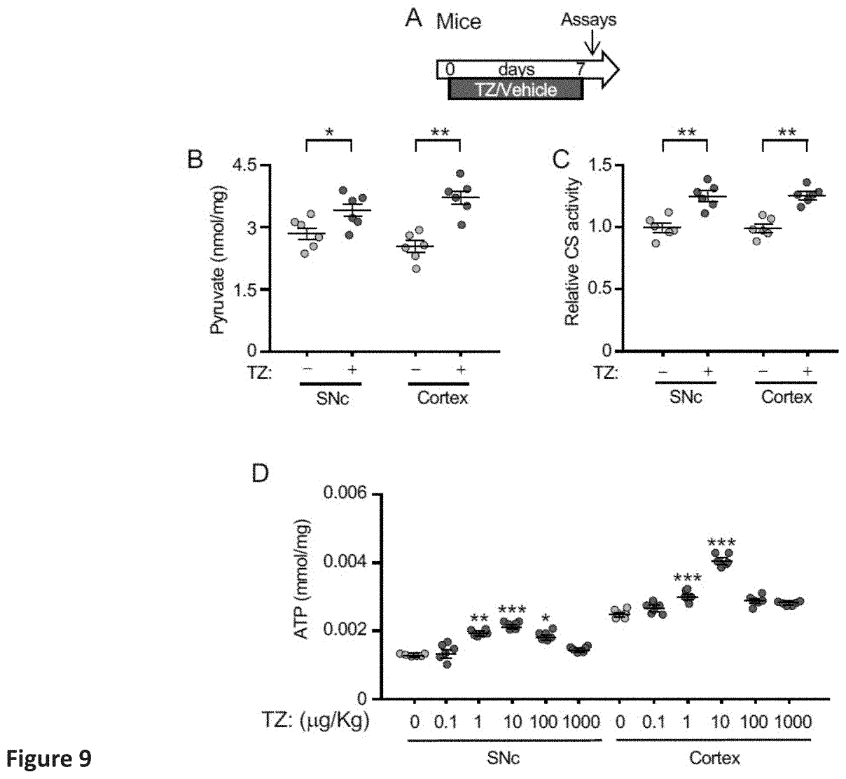

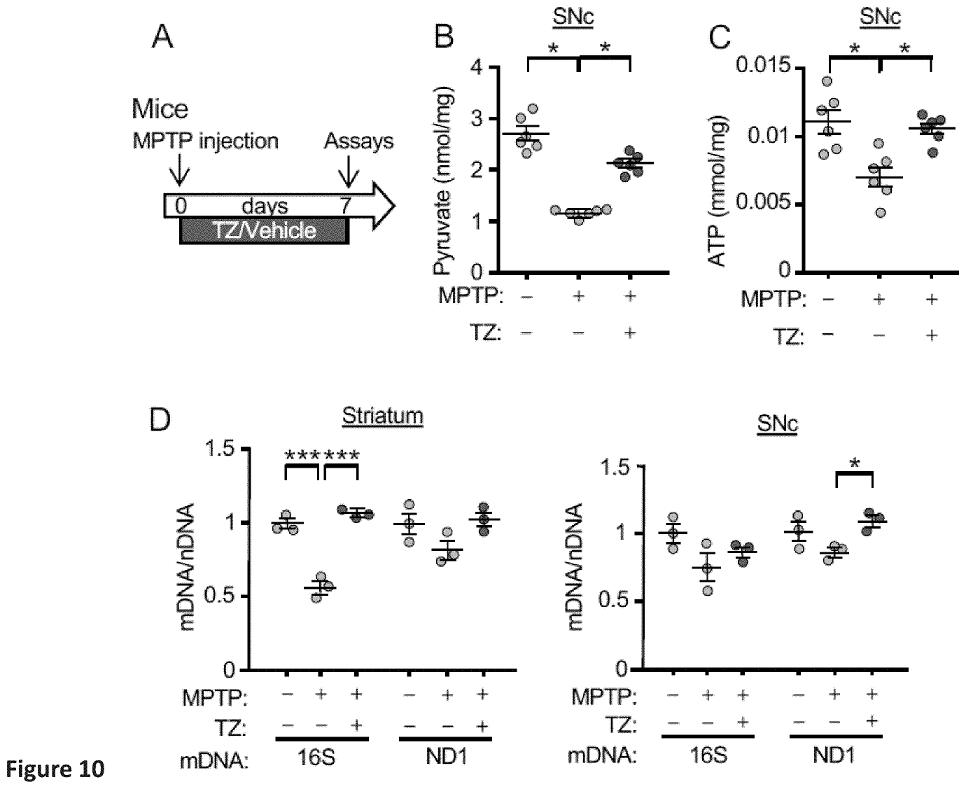

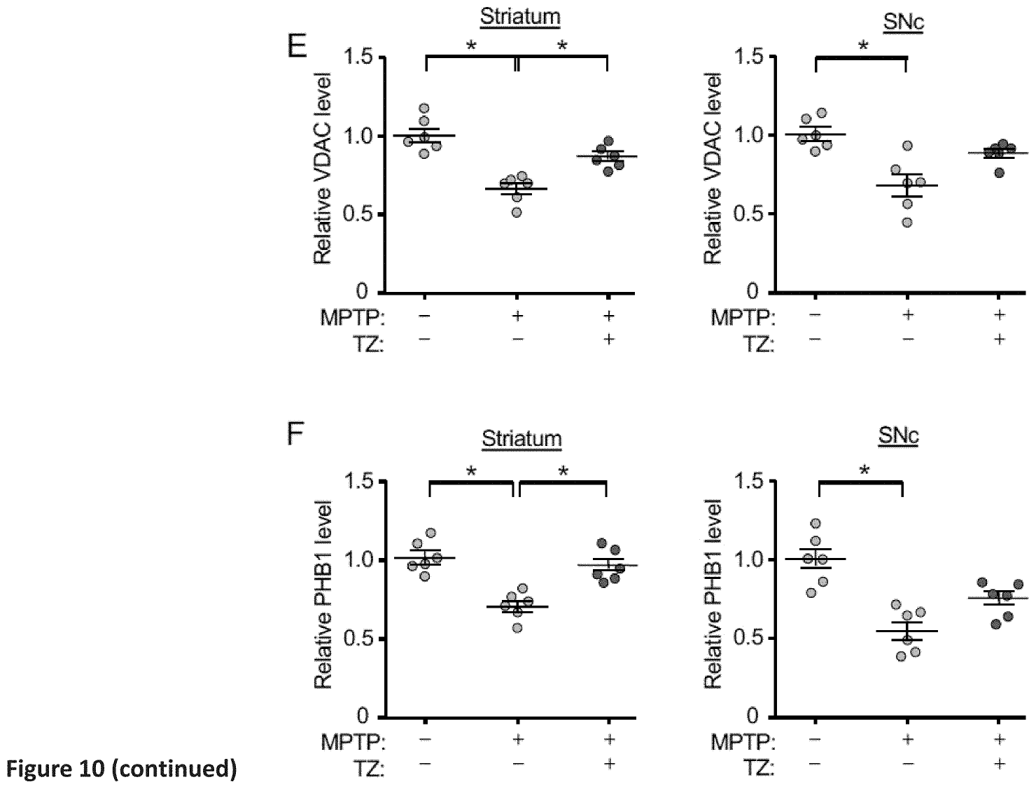

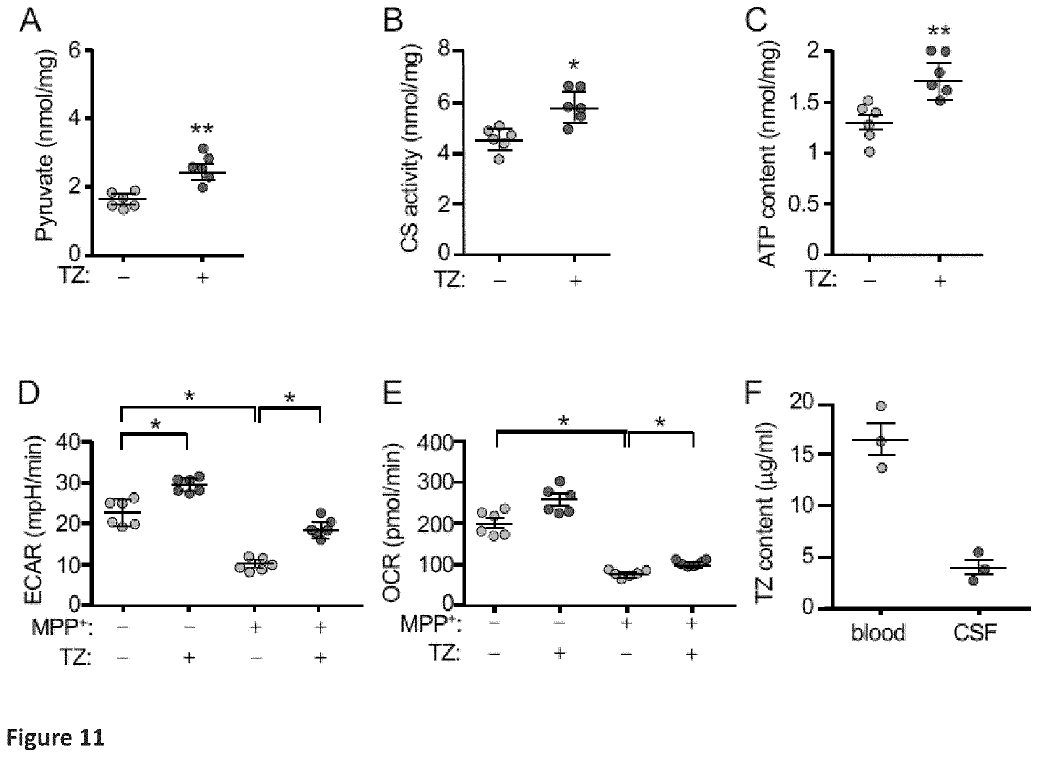

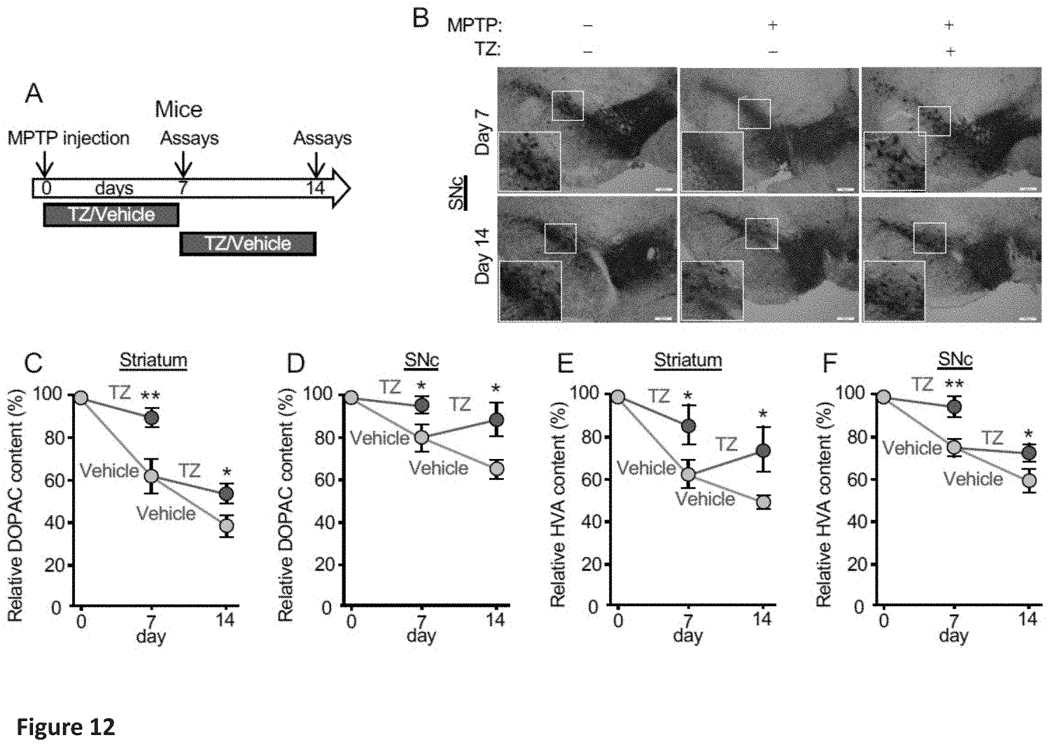

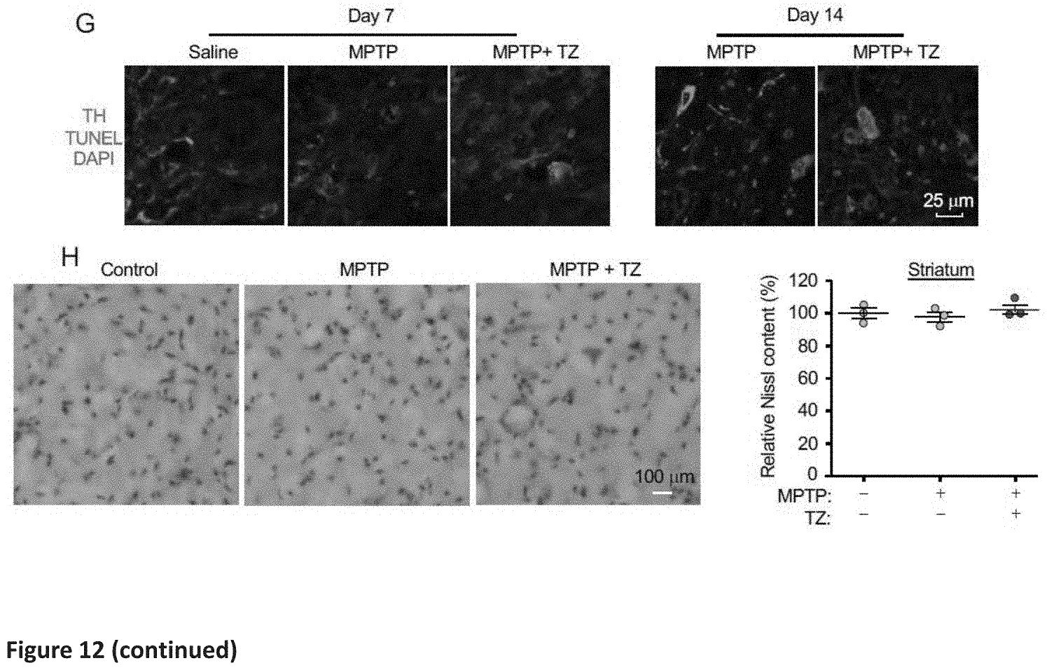

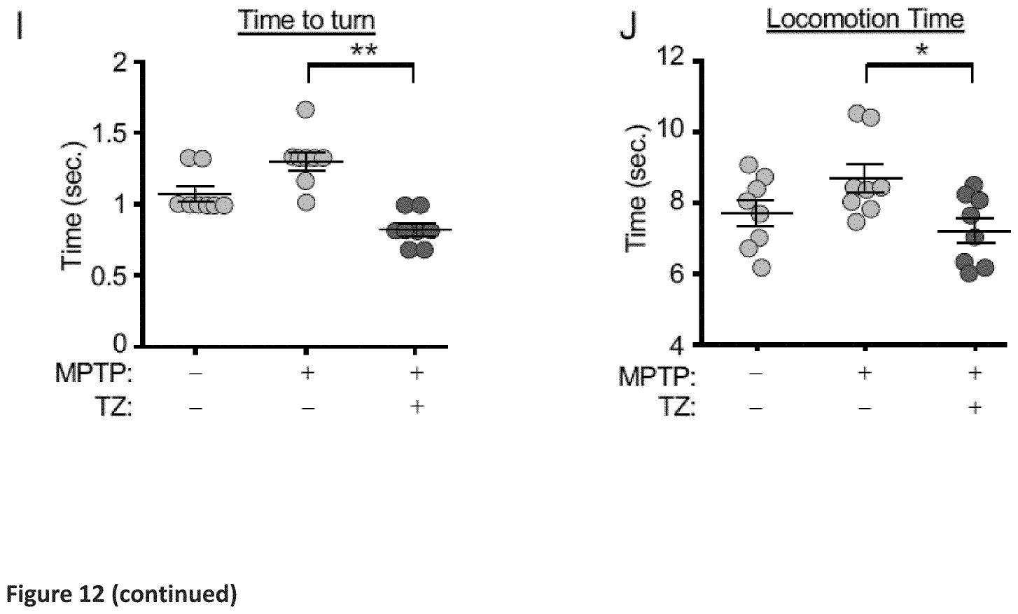

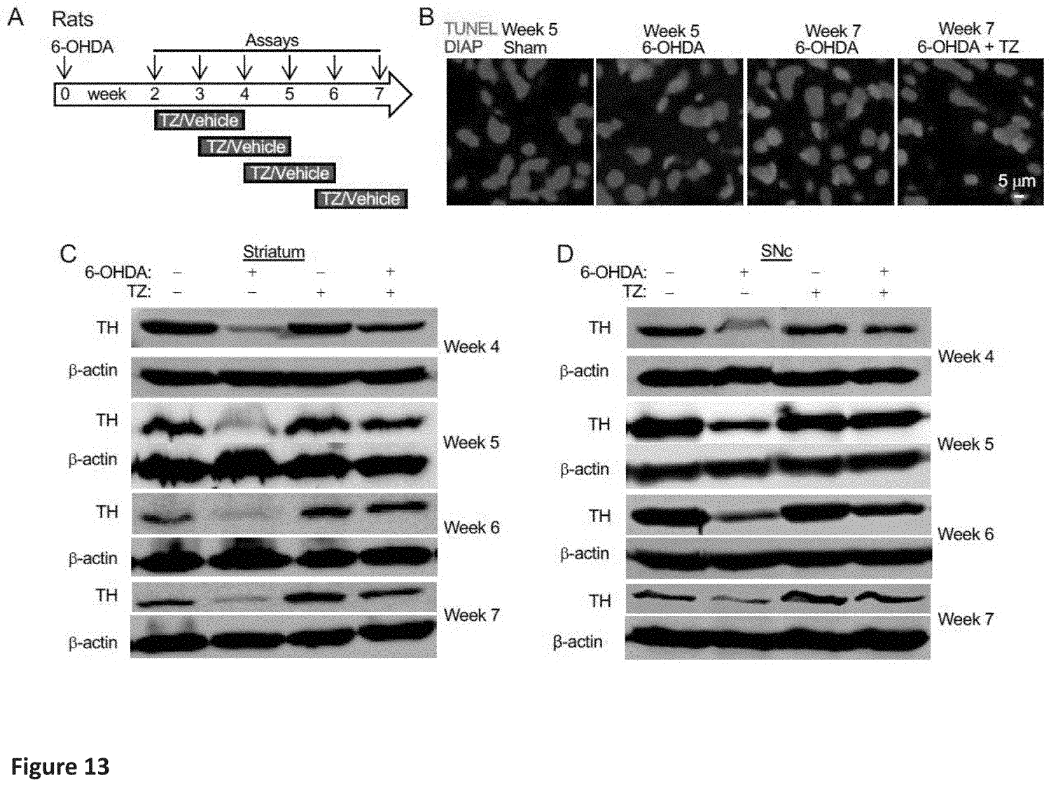

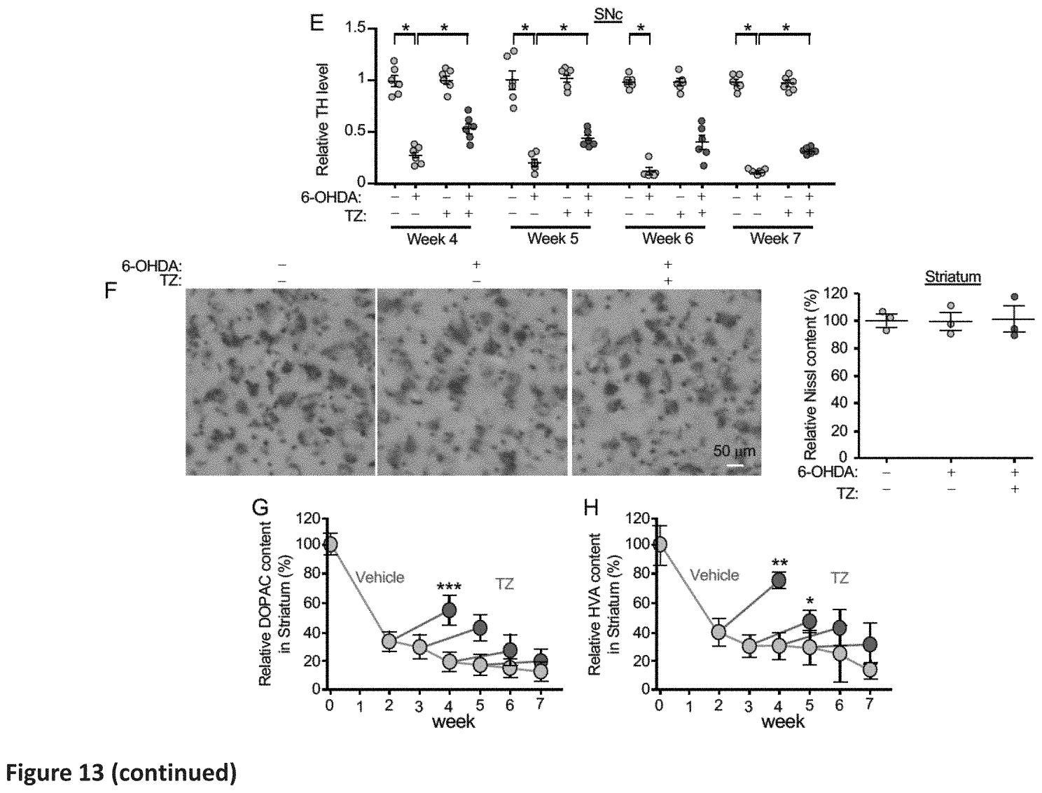

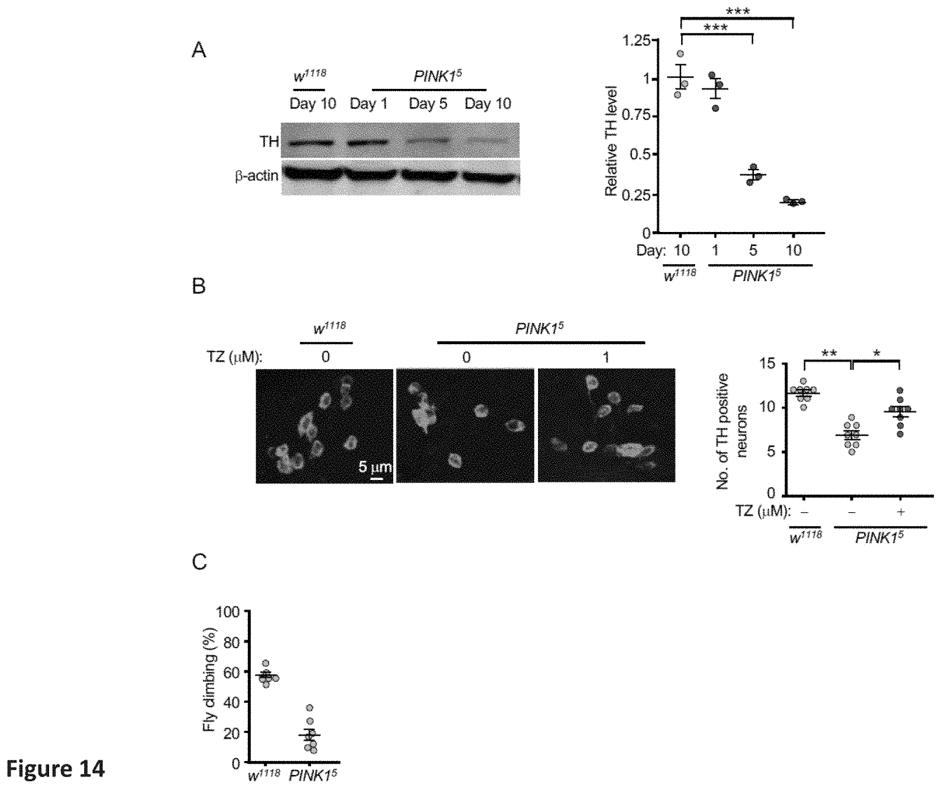

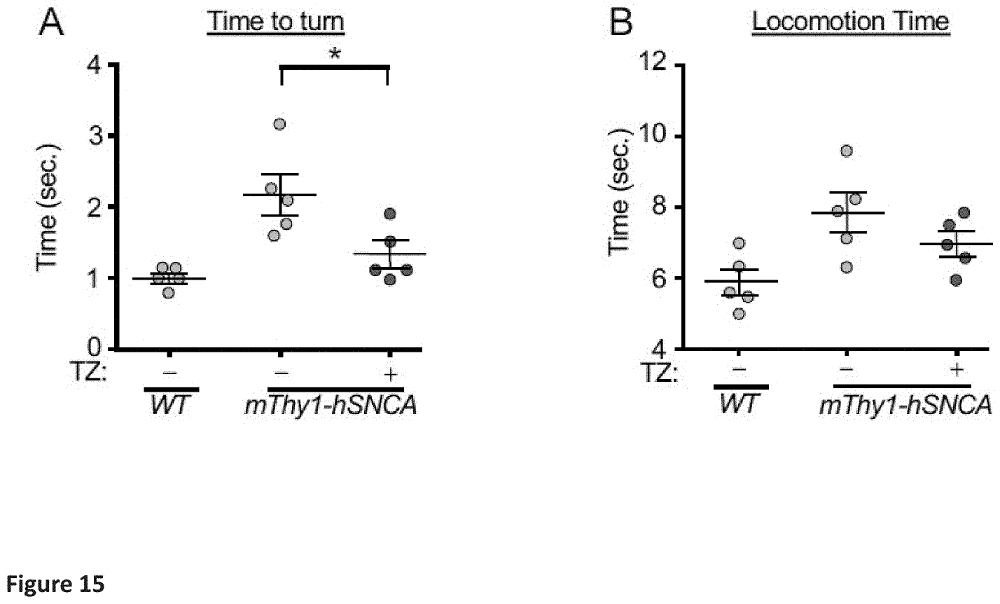

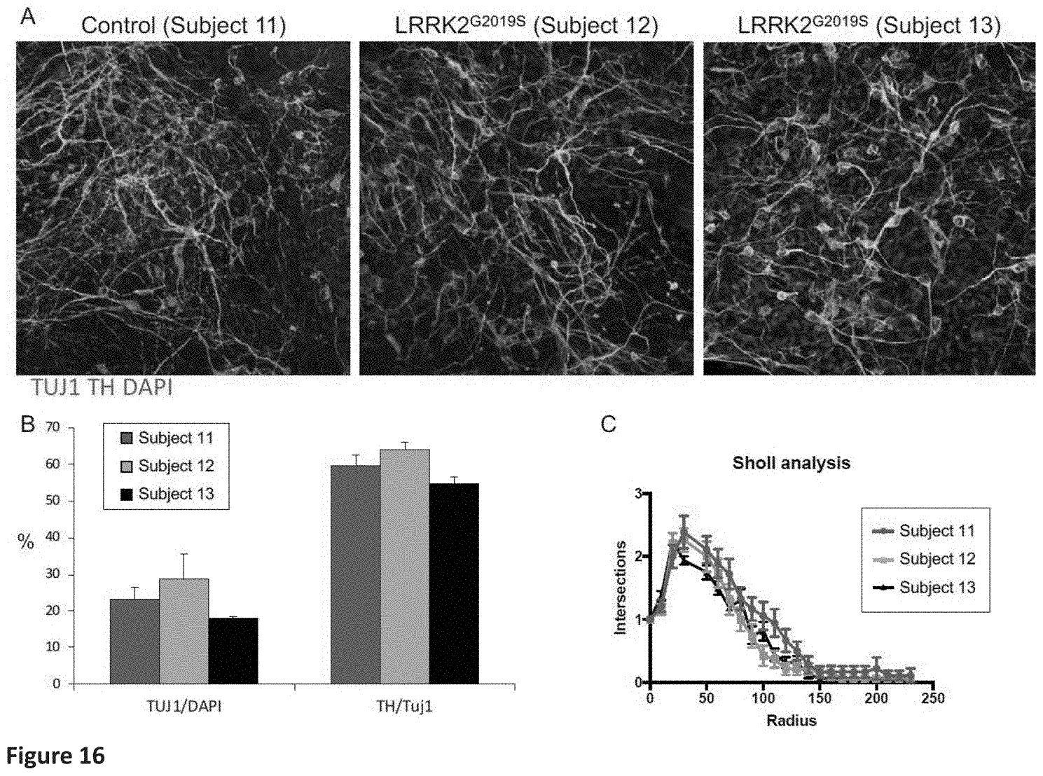

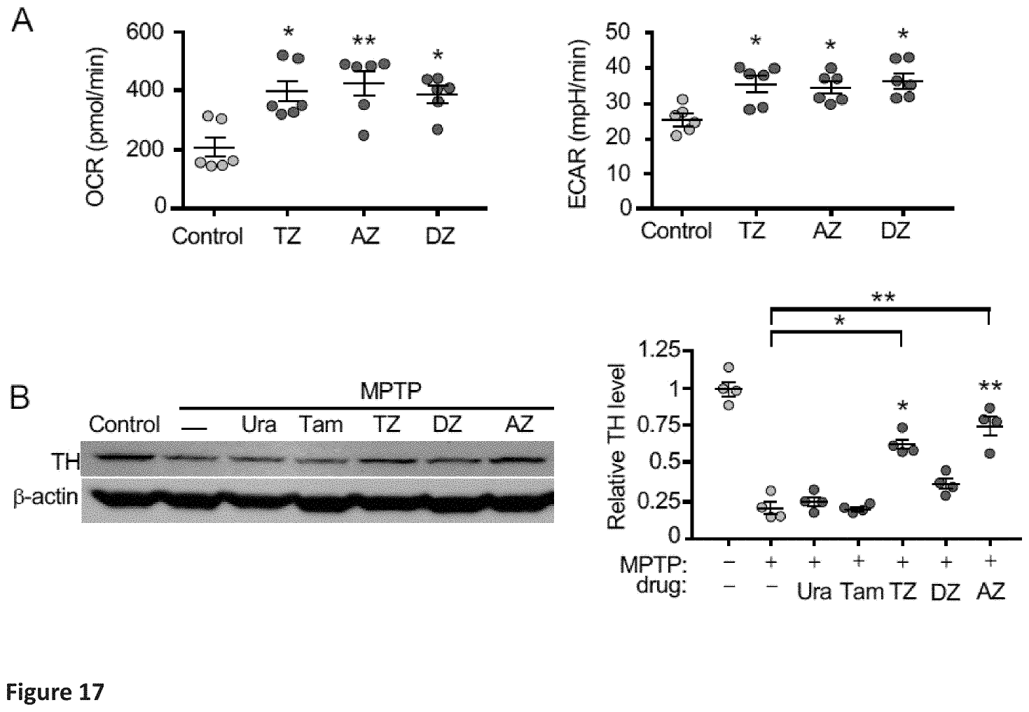

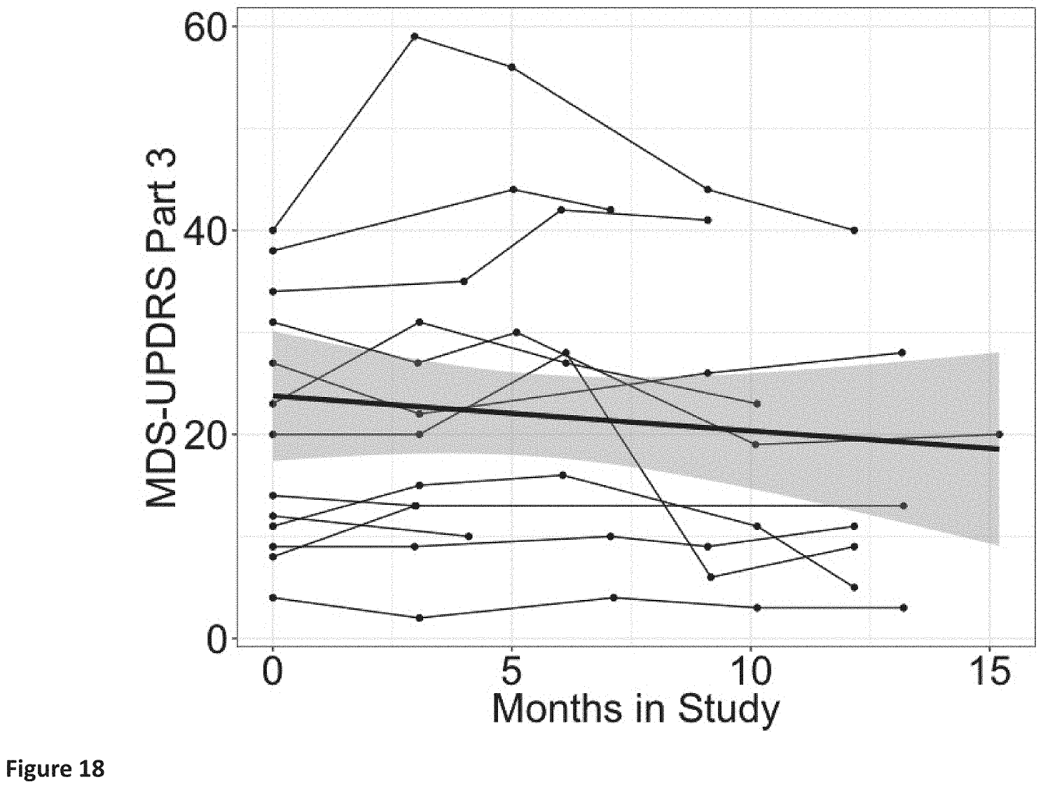

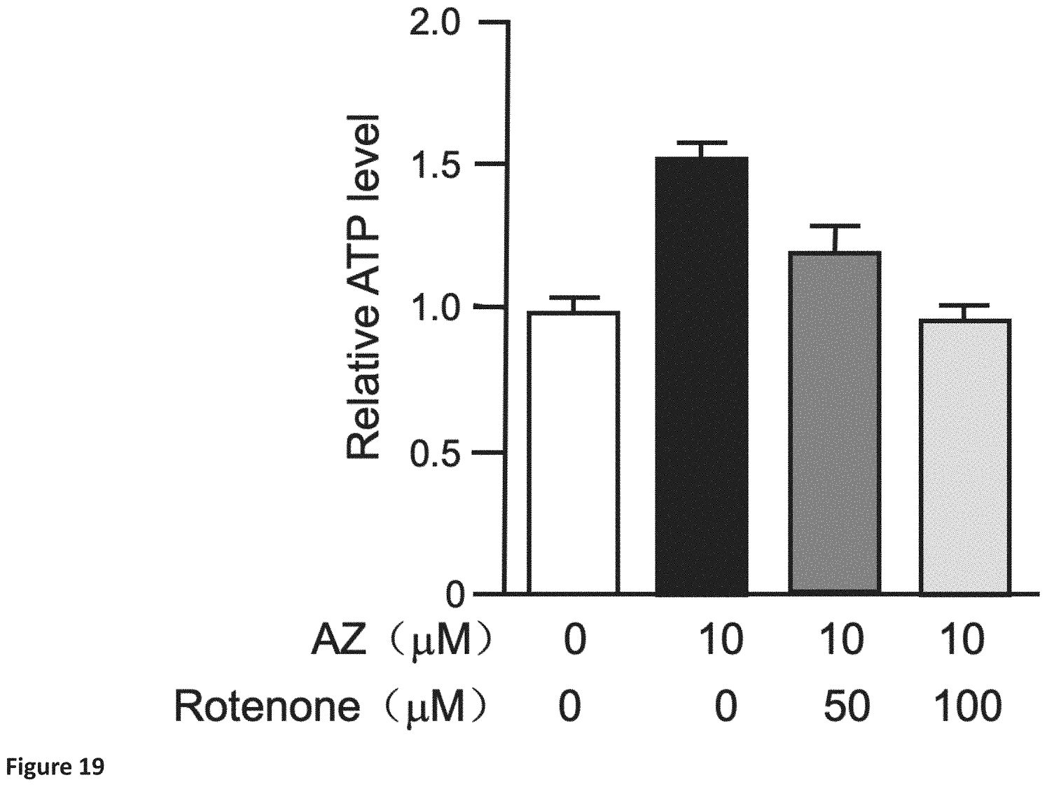

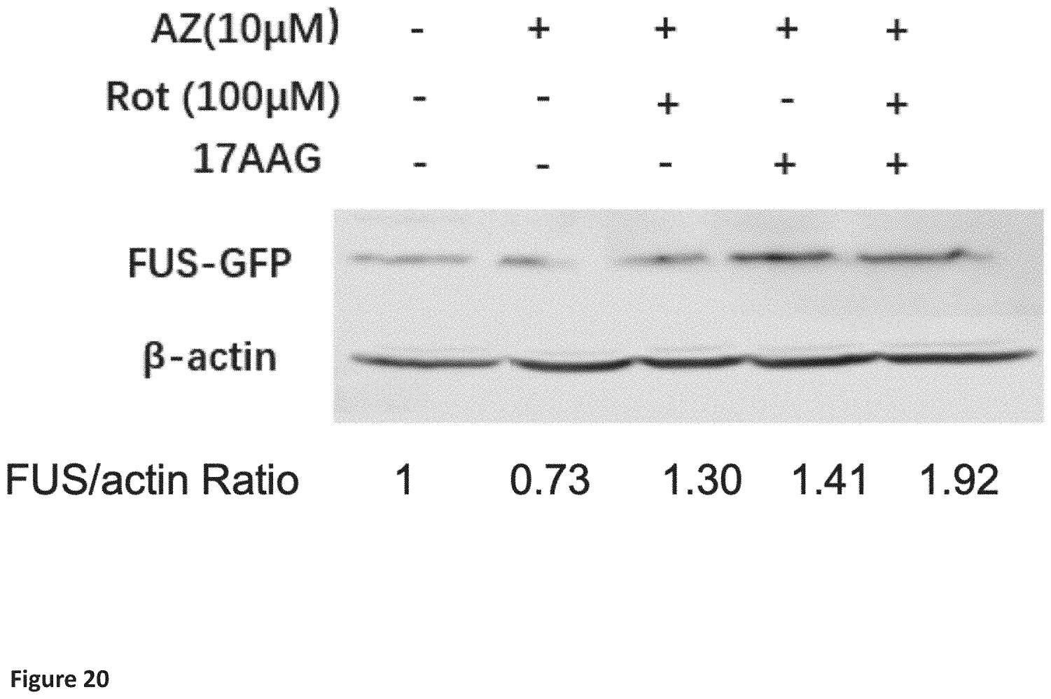

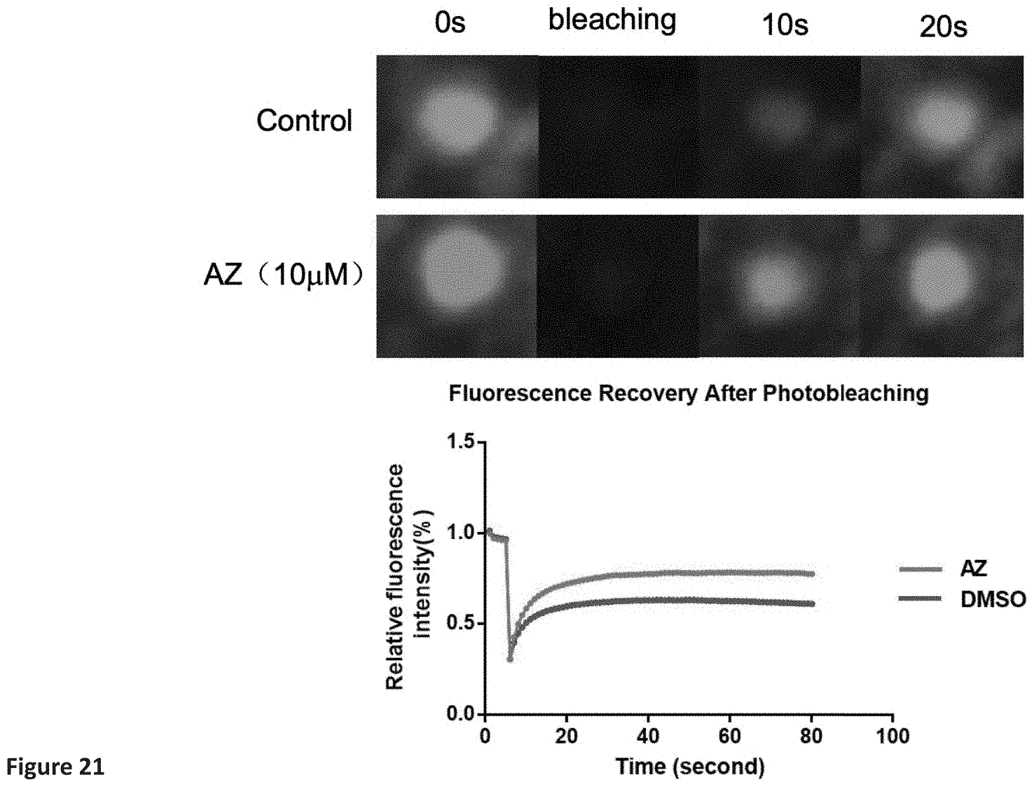

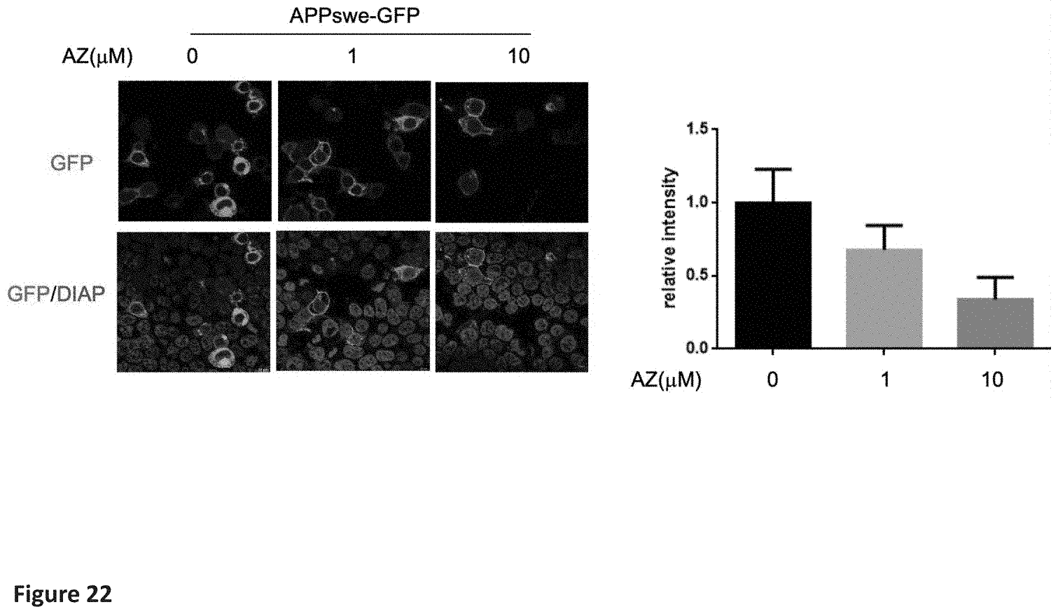

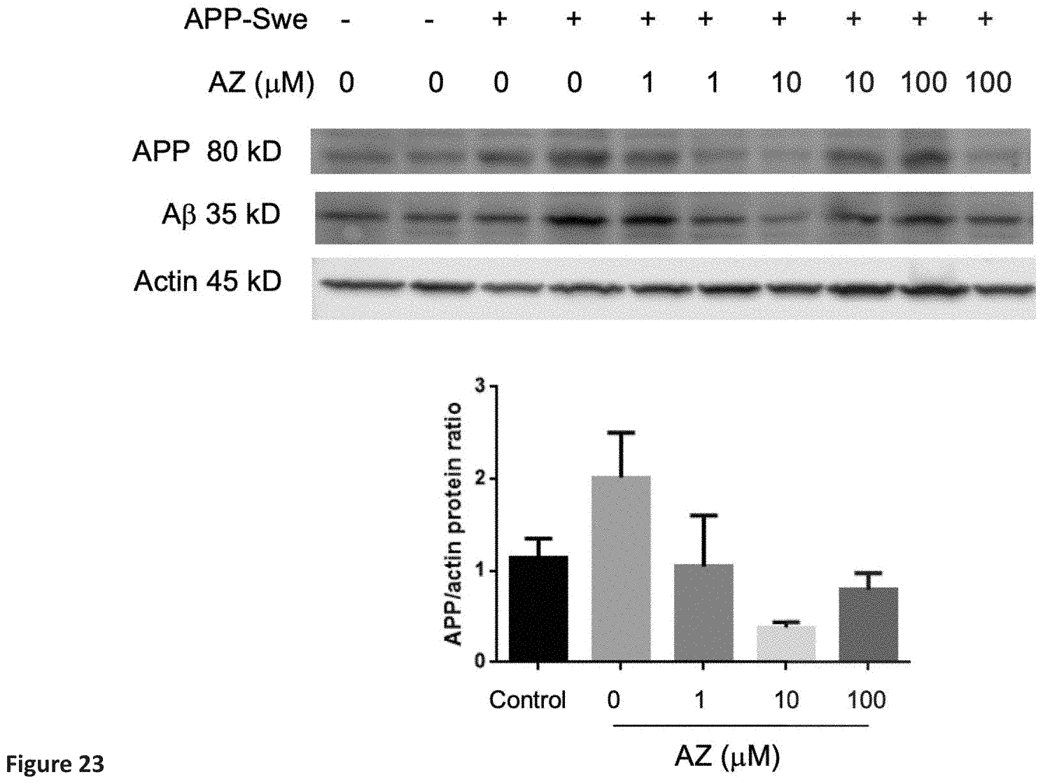

FIG. 9. TZ enhances glycolysis and mitochondrial function in vivo in mouse brain. In all figures, data points are from individual mice, rats, or groups of flies. Bars and whiskers indicate mean±SEM. Blue indicates controls and red indicates TZ treatment. In the figures, *p<0.05, **p<0.01, ***p<0.001. A) Schematic for experiments in panels B-D. Eight week-old C57bl/6 mice were given TZ (10 μg/kg) or vehicle and were injected i.p. once a day for one week. Assays were at day 7. Samples are from the same animals shown in FIG. 1C-1E. B-D) Pyruvate levels (B), citrate synthase (CS) activity (C), and ATP levels (D) measured in mouse SNc and cortex. In panel D, TZ doses are indicated. N=6. Statistical comparison is vs. 0 TZ.

FIG. 10. TZ decreases MPTP-induced reductions in glycolysis, ATP levels, and mitochondrial defects in mice. A) Schematic of experiments in panels B-F. C57BL/6 mice (8 week-old) received 4 i.p. injections of MPTP (20 mg/kg at 2 hr intervals) or vehicle on day 0. Mice were then injected with TZ (10 μg/kg) or vehicle (0.9% saline) once a day for one week and assays were performed on day 7. Samples are from the same animals shown in FIG. 2E,2F. B,C) Pyruvate and ATP levels in mouse SNc. N=6. D) Mitochondrial DNA (mDNA, 16S and ND1) relative to nuclear DNA (nDNA, intron β-globin) by quantitative PCR of mouse striatum and SNc. N=3. E,F) VDAC (E) and PHB1 (F) protein levels in striatum and SNc assayed by western blot and normalized to control. N=6.

FIG. 11. TZ enhances glycolysis and mitochondrial function in M17 human neuroblastoma cells. A-C) M17 cells were treated with TZ (10 μM) or vehicle. Pyruvate levels (A), citrate synthase (CS) activity (B), and ATP levels (C) were measured 24 hr later. N=6. D-E) M17 cells were treated for 24 hr with vehicle or the MPTP metabolite, 1-methyl-4-phenylpyridinium (MPP⁺), which inhibits mitochondrial complex I respiration. They also received TZ (10 μM) or vehicle. Basal extracellular acidification rate (ECAR), a measure of glycolysis (D), and basal 02 consumption rate (OCR), a measure of mitochondrial respiration (E), were measured 24 h after TZ treatment. N=6. F) TZ levels in blood and cerebral spinal fluid. TZ was injected i.p. at 30 mg/kg. Blood and cerebrospinal fluid were collected 20 min. later. TZ was quantified by HPLC-ECD. This dose of TZ is substantially higher than that used to activate glycolysis; we used that dose in order to readily detect TZ in the blood and cerebral spinal fluid. Although the mice appeared healthy with this dose, we cannot exclude some adverse effect. N=3.

FIG. 12. TZ attenuated TH-positive neuron death and improved function in an MPTP mouse model. A) Schematic for experiments in panels B-J. C57BL/6 mice (8 week-old) received 4 i.p. injections of MPTP (20 mg/kg at 2 hr intervals) or vehicle on day 0. Mice were then injected with TZ (10 μg/kg) or vehicle (0.9% saline) once a day for one week and assays were performed on day 7. Other mice began receiving daily TZ or vehicle injections beginning on day 7 and assays were performed on day 14. Protocol is same as shown in FIG. 2. B) Example of TH immunostaining in the SNc on days 7 and 14. Inset shows areas that are shown in FIG. 2E. Scale bar, 500 μm. C-F) Measurement of DOPAC in mouse striatum (C) and SNc (D) and measurement of HVA in striatum (E) and SNc (F). (N=6). G) Example of TH and TUNEL co-staining in the SNc. TH (green), TUNEL (red), and DAPI (nuclei, blue). Scale bar, 25 μm. Quantitative data are in FIG. 2J. H) Left panels are Nissl staining of neurons in the striatum. Samples were obtained 7 days after MPTP injection. Right panels show the quantification. Results showed no reduction of total number of neurons in the striatum after MPTP injection, indicating lack of substantial cell death except in dopamine neurons. Scale bar, 400 μm. N=3 per group. I,J) Behavioral response of mice in the pole test. (I) Time mice took to turn their heads from upward to downward. (J) Time mice took to climb down the pole. N=8.

FIG. 13. TZ attenuates neurodegeneration, increases TH and dopamine, and improves motor function when administered after the onset of deterioration. A) Schematic for experiments in panels B-H. 6-OHDA (20 μg) was injected into right striatum of rats on day 0. TZ (70 μg/kg) or saline were injected (i.p.) daily for 2 weeks, beginning 2, 3, 4, or 5 weeks after 6-OHDA injection. Assays were at 0 and 2-7 weeks. Protocol is same as shown in FIG. 3. B) Example of TUNEL staining in the SNc of rat brain. Samples were obtained at 5 weeks in sham-treated animals, 5 weeks in animals that received 6-OHDA, and at 7 weeks in animals that received vehicle or TZ from week 5 to 7. TUNEL (red) and DAPI (nuclei, blue). Scale bar, 5 μm. C-E) Examples of western blots of TH and β-actin (protein loading control) in the striatum (C) and SNc (D). (E) shows quantification for SNc; quantification for striatum is in FIG. 3C. F) Nissl staining of neurons in the striatum region. Samples were obtained 2 weeks after 6-OHDA injection. Right panels show the quantification. Results showed no obvious reduction of total number of neurons in the striatum, indicating lack of substantial cell death except in dopamine neurons. Scale bar, 50 μm. Right panels show the quantification. N=3 per group. G,H) Measurement of DOPAC (G) and HVA (H) in right striatum relative to left (control) striatum. N=6.

FIG. 14. A genetic model of PD in PINK1⁵ flies. A) TH levels in the brain of PINK1⁵ flies. Left panel shows example of western blot on the 1^{st}, 5^{th}, and 10^{th} day after hatching. β-actin is protein loading control. Right panel shows quantification. N=3 with 40 fly heads for each treatment in each trial. B) Immunostaining for TH in PINK1⁵ fly brain PPL1 cluster. Left panel shows example of immunostaining for TH. W¹¹¹⁸ flies were used as a genetic background matched control. Quantification of TH neurons is on the right. N=8. C) Climbing assay for day 1 after eclosion. Note that by day 1 motor performance is already markedly degraded. N=3, with 100 flies for each treatment in each trial.

FIG. 15. TZ improves motor performance in mThy-hSNCA mice. Performance of 15 month-old mThyl-hSNCA transgenic mice in the pole test. A) The time mice took to turn their heads from upward to downward. B) The time mice took to climb down the pole. Five mice were tested for each condition.

FIG. 16. iPSC-derived dopamine neurons from patients with LRRK2^{G2019S}. A) Example of immunofluorescence images of human iPSC-derived DA neurons from a healthy individual (Control, Subject 11), and two independent patients with PD (Subject 12 and 13) carrying the LRRK2G2019S mutation. After 30 days of differentiation, the data showed comparable extents of differentiation and absence of neurodegeneration phenotypes in PD samples. Green labels neuron marker TUJ1, red labels TH, and blue is DAPI (nuclei). Scale bar, 50 μm. B) Data are the percentage of total neurons (TUJ1/DAPI) and the percentage of neurons that are TH positive (TH/TUJ1). C) Sholl analysis of TH positive neurons

FIG. 17. Terazosin, doxazosin, and alfuzosin (TZ/DZ/AZ) enhance glycolysis and mitochondrial function in M17 human neuroblastoma cells and TH levels in MPTP-treated mice. A) Basal O₂ consumption rate (OCR), a measure of mitochondrial respiration, and basal extracellular acidification rate (ECAR), a measure of glycolysis, were measured 24 hr after adding TZ (10 μM), doxazosin (10 μM), or alfuzosin (10 μM) to M17 human neuroblastoma cells. N=6. Statistical comparisons are to control. B) Example of western blot of TH and β-actin (protein loading control) in SNc. Quantification is shown on the right. TH protein levels were normalized to the control. Statistical comparisons are to MPTP alone. N=4.

FIG. 18. UPDRS scores for 13 patients with PD taking TZ/DZ/AZ. Each set of data points and lines indicates an individual patient. Bold line and shading indicate the linear regression line and 95% confidence intervals for the 13 patients. See legend of FIG. 7 for more information.

FIG. 19. Relative ATP levels in Hela cells expressing FUS-GTP after treatment with alfazosin (AZ) and rotenone (Rot).

FIG. 20. Expression levels of FUS-GTP in cells treated with alfazosin (AZ), rotenone (Rot), and 17AAG, an inhibitor of the ATPase of HSP90.

FIG. 21. Fluorescence recovery of FUS-GTP in Hela cells after photobleaching.

FIG. 22. Expression of amyloid precursor protein (APP) Swedish mutation tagged with GFP (APPswe-GFP) in transfected Hek293T cells. Left panel—expression of GFP in cells treated with alfazosin (AZ). Right panel—relative intensity versus treatment with increasing concentrations of AZ.

FIG. 23. Western blot quantification of APPswe-GFP in transfected Hek293T cells treated with increasing concentration of alfazosin (AZ).

### DETAILED DESCRIPTION

The disclosed subject matter further may be described utilizing terms as defined below.

Unless otherwise specified or indicated by context, the terms “a”, “an”, and “the” mean “one or more.” For example, “a modulator of phosphoglycerate kinase 1 (PGK1) activity” should be interpreted to mean “one or more modulators of phosphoglycerate kinase 1 (PGK1) activity.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus <10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

The terms “subject,” “patient,” and “individual” may be used interchangeably herein. A subject may be a human subject. A subject may refer to a human subject having or at risk for acquiring a disease or disorder that is associated with phosphoglycerate kinase 1 (PGK1) activity and/or that may be treated and/or preventing by modulating the activity of PGK1.

As used herein, the term “modulate” means decreasing or inhibiting activity and/or increasing or augmenting activity. For example, modulating PGK1 activity may mean increasing or augmenting PGK1 activity and/or decreasing or inhibiting PGK1 activity. The therapeutic agents disclosed herein may be administered to modulate PGK1 activity. The methods disclosed herein may include administering to a subject in need thereof a pharmaceutical composition comprising an effective amount of a therapeutic agent that activates PGK1.

Diseases and disorders treated and/or prevented by the methods disclosed herein include diseases or disorders that may be treated and/or prevented by modulating the activity of PGK1, which may include neurodegenerative diseases and disorders and symptoms thereof. Neurodegenerative diseases and disorders may include, but are not limited to, Parkinson's disease (PD), Alzheimer's disease (AD), Huntington's disease (HD), amyotrophic lateral sclerosis (ALS), and Lewy body dementia, and symptoms of neurodegenerative diseases and disorders may include, but are not limited to, sleep disturbances, depression, and weakness.

As used herein, the phrase “effective amount” shall mean that drug dosage that provides the specific pharmacological response for which the drug is administered in a significant number of patients in need of such treatment. An effective amount of a drug that is administered to a particular patient in a particular instance will not always be effective in treating the conditions/diseases described herein, even though such dosage is deemed to be a therapeutically effective amount by those of skill in the art.

Chemical Compounds

The presently disclosed methods and compositions include and/or utilized therapeutic agents which may include chemical compounds, which otherwise may be referred to as small molecules. The chemical compounds may be described using terminology known in the art and further discussed below.

As used herein, an asterick “*” or a plus sign “+” may be used to designate the point of attachment for any radical group or substituent group.

The term “alkyl” as contemplated herein includes a straight-chain or branched alkyl radical in all of its isomeric forms, such as a straight or branched group of 1-12, 1-10, or 1-6 carbon atoms, referred to herein as C1-C12 alkyl, C1-C10-alkyl, and C1-C6-alkyl, respectively.

The term “alkylene” refers to a diradical of an alkyl group (e.g., —(CH₂)ₙ— where n is an integer such as an integer between 1 and 20). An exemplary alkylene group is —CH₂CH₂—.

The term “haloalkyl” refers to an alkyl group that is substituted with at least one halogen. For example, —CH₂F, —CHF₂, —CF₃, —CH₂CF₃, —CF₂CF₃, and the like.

The term “heteroalkyl” as used herein refers to an “alkyl” group in which at least one carbon atom has been replaced with a heteroatom (e.g., an O, N, or S atom). One type of heteroalkyl group is an “alkoxy” group.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C2-C12-alkenyl, C2-C10-alkenyl, and C2-C6-alkenyl, respectively.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C2-C12-alkynyl, C2-C10-alkynyl, and C2-C6-alkynyl, respectively.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C4-8-cycloalkyl,” derived from a cycloalkane. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halo, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloalkylene” refers to a cycloalkyl group that is unsaturated at one or more ring bonds.

The term “partially unsaturated carbocyclyl” refers to a monovalent cyclic hydrocarbon that contains at least one double bond between ring atoms where at least one ring of the carbocyclyl is not aromatic. The partially unsaturated carbocyclyl may be characterized according to the number of ring carbon atoms. For example, the partially unsaturated carbocyclyl may contain 5-14, 5-12, 5-8, or 5-6 ring carbon atoms, and accordingly be referred to as a 5-14, 5-12, 5-8, or 5-6 membered partially unsaturated carbocyclyl, respectively. The partially unsaturated carbocyclyl may be in the form of a monocyclic carbocycle, bicyclic carbocycle, tricyclic carbocycle, bridged carbocycle, spirocyclic carbocycle, or other carbocyclic ring system. Exemplary partially unsaturated carbocyclyl groups include cycloalkenyl groups and bicyclic carbocyclyl groups that are partially unsaturated. Unless specified otherwise, partially unsaturated carbocyclyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the partially unsaturated carbocyclyl is not substituted, i.e., it is unsubstituted.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using 5 Cx-Cx nomenclature where x is an integer specifying the number of ring atoms. For example, a C3-C7 heterocyclyl group refers to a saturated or partially unsaturated 3- to 7-membered ring structure containing one to four heteroatoms, such as nitrogen, oxygen, and sulfur. The designation “C3-C7” indicates that the heterocyclic ring contains a total of from 3 to 7 ring atoms, inclusive of any heteroatoms that occupy a ring atom position.

The terms “amine” and “amino” are art-recognized and refer to both unsubstituted and substituted amines (e.g., mono-substituted amines or di-substituted amines), wherein substituents may include, for example, alkyl, cycloalkyl, heterocyclyl, alkenyl, and aryl.

The terms “alkoxy” or “alkoxyl” are art-recognized and refer to an alkyl group, as defined above, having an oxygen radical attached thereto. Representative alkoxy groups include methoxy, ethoxy, tert-butoxy and the like.

An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, and the like.

The term “carbonyl” as used herein refers to the radical —C(O)—.

The term “oxo” refers to a divalent oxygen atom —O—.

The term “carboxamido” as used herein refers to the radical —C(O)NRR′, where R and R′ may be the same or different. R and R′, for example, may be independently alkyl, aryl, arylalkyl, cycloalkyl, formyl, haloalkyl, heteroaryl, or heterocyclyl.

The term “carboxy” as used herein refers to the radical —COOH or its corresponding salts, e.g. —COONa, etc.

The term “amide” or “amido” or “amidyl” as used herein refers to a radical of the form —R¹C(O)N(R²)—, —R¹C(O)N(R²)R³—, —C(O)NR²R³, or —C(O)NH₂, wherein R¹, R² and R³, for example, are each independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” or “+” or “−” depending on the configuration of substituents around the stereogenic carbon atom and or the optical rotation observed. The present invention encompasses various stereo isomers of these compounds and mixtures thereof. Stereoisomers include enantiomers and diastereomers. Mixtures of enantiomers or diastereomers may be designated (±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise. Also contemplated herein are compositions comprising, consisting essentially of, or consisting of an enantiopure compound, which composition may comprise, consist essential of, or consist of at least about 50%, 60%, 70%, 80%, 90%, 95%, 96%, 97%, 98%, 99%, or 100% of a single enantiomer of a given compound (e.g., at least about 99% of an R enantiomer of a given compound).

Methods of Treating Neurodegenerative Diseases Using Modulators of Phosphoglycerate Kinase 1 (PGK1) Activity

The subject matter of the application relates to methods and compositions for treating and/or preventing a neurodegenerative diseases or disorders or symptoms thereof in a subject in need thereof. The methods may include administering to the subject a pharmaceutical composition comprising an effective amount of a therapeutic agent that binds and/or activates phosphoglycerate kinase 1 (PGK1). Suitable neurodegenerative diseases or disorders that may be treated by the disclosed methods and compositions may include, but are not limited to Parkinson's disease (PD), Alzheimer's disease (AD), Huntington's disease, amyotrophic lateral sclerosis (ALS), and Lewy body dementia, and symptoms thereof may include, but are not limited to, sleep disturbances, depression, and weakness.

In some embodiments, the methods and compositions are utilized for treating and/or preventing a neurodegenerative disease or disorder or symptoms thereof in a subject in need thereof selected from the group consisting of Alzheimer's disease (AD), Huntington's disease (HD), amyotrophic lateral sclerosis (ALS), Lewy body dementia, the method comprising administering to the subject a pharmaceutical composition comprising an effective amount of a therapeutic agent that activates phosphoglycerate kinase 1 (PGK1) selected from the group consisting of terasozin, prazosin, doxozosin, alfuzosin, trimazosin, and abanoquil or pharmaceutical salts or hydrates thereof.

In the disclosed methods and compositions, the therapeutic agent utilized in the disclosed methods and compositions may be a compound (or a small molecule) that binds to PGK1. In some embodiments, the therapeutic agent is a compound that binds to PGK1 with a dissociation constant (K_{d}(PGK1)) of less than about 10 μM, 5 μM, 2 μM, 1 μM, 0.5 μM, 0.2 μM, 0.1 μM, 0.05 μM, 0.02 μM, or 0.01 μM and activates PGK1.

In some embodiments of the disclosed methods and compositions, the compound does not bind to the α₁-adrenergic receptor (α₁AR) and/or does not function as a ligand for the α₁AR as an agonist or antagonist. If the compound binds to the α₁AR, preferably the compound binds to the α₁AR with a dissociation constant (K_{d}(α₁AR)) greater than about 10 μM, 20 μM, 50 μM, 100 μM, 200 μM, 500 μM, or 1000 μM. In some embodiments, where the compound binds to PGK1 and to α₁AR the ratio K_{d}(PGK1)/K_{d}(α₁AR) is greater than about 10, 50, 100, 500, 1000, 5000, 10000, or higher.

In the disclosed methods and compositions, the therapeutic agent may be selected from compounds characterized as having a substituted isoquinoline core or a substituted quinazoline core. In some embodiments, the compounds may be characterized as a having a diamino-substituted isoquinoline core (e.g., a 1,3-diaminoisoquinoline core) or a diamino-substituted quinazoline core (e.g., a 2,4-diaminoquinazoline core), which may be further substituted In further embodiments, the compounds may have an amino, piperazinyl-substituted core (e.g. a 1-amino, 3-N-piperazinyl-isoquinoline core, which may be further substituted, or a 2-N-piperazinyl, 4-aminoquinazoline core, which may be further substituted). Isoquinoline derivatives and quinazoline derivatives and their methods of synthesis are described in the art. (See, e.g., Chen et al., Nat. Chem. Biol. 2015 January; 11(1):19-25; and Bordner, J. Med. Chem. 1988, 31, 1036-1039; the contents of which are incorporated herein by reference in their entireties).

In some embodiments of the disclosed methods and compositions, the therapeutic agent is a compound having the following formula or a salt or hydrate thereof:

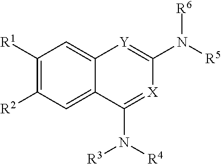

wherein:

X and Y are independently selected from CH and N, preferably at least one of X and Y is N; more preferably at least X is N; even more preferably X is N and Y is CH;

R¹ and R² are independently selected from hydrogen, alkyl, alkoxy, halo, alkylhalo, amino, cyano, and phenyl.

R³ and R⁴ are independently selected from hydrogen and alkyl;

R⁵ and R⁶ are independently selected from hydrogen, alkyl, or

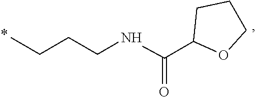

or R⁵ and R⁶ form a 5-membered or 6-membered homocycle or heterocycle (or two fused 5-membered or 6-membered homocycles or heterocycles) which is saturated or unsaturated at one or more bonds and optionally is substituted to include one or more non-hydrogen substituents, which non-hydrogen substituents optionally are selected from alkyl, halo, haloalkyl, hydroxyl, phenyl, amino, and carbonyl, and in particular R⁵ and R⁶ may form piperazinyl or a substituted piperazinyl, and optionally R⁵ and R⁶ form substituted piperazinyl having a formula

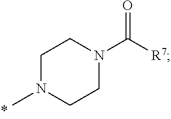

R⁷ is alkyoxy, or R⁷ is a one 3-membered ring, one 4-membered ring, one 5-membered ring, one 6-membered ring, or one 7-membered ring which ring is optionally saturated or unsaturated, or R⁷ is two fused rings which may be 5-membered rings or 6-membered rings which rings are optionally saturated or unsaturated, which one ring or two fused rings are carbocycles or heterocycles including one or more heteroatoms, which one ring or two fused rings optionally are substituted to include one or more non-hydrogen substituents, which non-hydrogen substituents optionally are selected from alkyl, halo, haloalkyl, hydroxyl, phenyl, amino, and carbonyl.

In further embodiments of the disclosed methods and compositions, the therapeutic agent is a compound having the following formula or a salt or hydrate thereof:

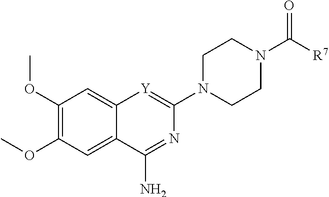

wherein:

Y is CH or N, and preferably Y is CH;

R⁷ is alkyoxy, or R⁷ is one 3-membered ring, one 4-membered ring, one 5-membered ring, one 6-membered ring, or one 7-membered ring which ring is optionally saturated or unsaturated, or R⁷ is two fused rings which may be 5-membered rings or 6-membered rings which rings are optionally saturated or unsaturated, which one ring or two fused rings are carbocycles or heterocycles including one or more heteroatoms, which one ring or two fused rings optionally are substituted to include one or more non-hydrogen substituents, which non-hydrogen substituents optionally are selected from alkyl, halo, haloalkyl, hydroxyl, phenyl, amino, and carbonyl.

In some embodiments of the disclosed methods and compositions, the therapeutic agent is selected from terazosin, prazosin, doxozosin, alfuzosin, trimazosin, and abanoquil or salts or hydrates thereof:

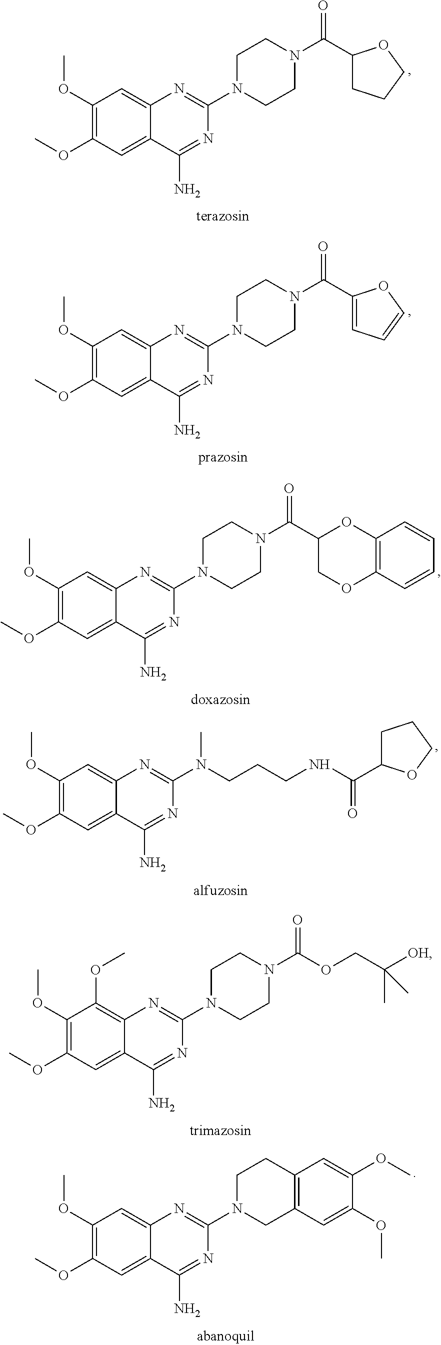

Phosphoglycerate Kinase 1 (PGK1) Activity Modulation

The compounds disclosed herein preferably modulate activity of phosphoglycerate kinase 1 (PGK1). Modulation may include activating or increasing PGK1 activity. However, modulation also may include inhibiting or decreasing PGK1 activity. PGK1 activity may be assessed utilizing methods known in the art and the methods disclosed herein, including the methods disclosed in the Examples provided herein. In some embodiments, the compounds decrease or increase PGK1 activity relative to a control (e.g., by at least about 10%, 20%, 30%, 40%, 50%, 60%, 70%, 80%, 90%, 100%, 200%, 300%, 400%, 500%, 600%, 700%, 800%, 900%, 1000% or more (or within a range bounded by any of these values)). In other embodiments, the compounds activate PGK1 greater than about 2-fold, 3-fold, 4-fold, 5-fold, 10-fold, 20-fold, 30-fold, 40-fold, 50-fold, 60-fold, 70-fold, 80-fold, 90-fold, or 100-fold, relative to a control. In other embodiments, the compounds activate PGK1 with a maximum activation (Eₘₐₓ) greater than about 100%, 200%, 300%, 400%, 500%, 600%, 700%, 800%, 900%, 1000%, 1100%, 1200%, 1300%, 1400%, or 1500% (or within a range bounded by any of these values). In other embodiments, an IC₅₀ value for the compound in regard to activation of PGK1 may be determined and preferably the compound has an IC₅₀ value of less than about 10 μM, 5 μM, or 1 μM, 0.5 μM, 0.1 μM, 0.05 μM, 0.01 μM, 0.005 μM, or 0.001 μM (or within a range bounded by any of these values).

Methods for measuring binding of compounds to PGK1 and PGK1 activity are known in the art. (See, e.g., Chen et al., Nat. Chem. Biol. 2015 January; 11(1):19-25; the content of which is incorporated herein by reference in its entirety).

In some embodiments, the compounds disclosed herein do not bind to the α₁-adrenergic receptor (α₁AR). If the compound binds to the α₁AR, preferably the compound binds to the α₁AR with a dissociation constant (K_{d}(α₁AR)) greater than about 10 μM, 20 μM, 50 μM, 100 μM, 200 μM, 500 μM, or 1000 μM. In some embodiments, where the compound binds to PGK1 and to α₁AR the ratio K_{d}(PGK1)/K_{d}(α₁AR) is greater than about 10, 50, 100, 500, 1000, 5000, 10000, or higher.

Methods for measuring the binding of compounds to α₁AR are known in the art. (See Bordner, J. Med. Chem. 1988, 31, 1036-1039; the content of which is incorporated herein by reference in it entirety).

Pharmaceutical Compositions and Methods of Administration

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures.

The compounds for use according to the methods of disclosed herein may be administered as a single compound or a combination of compounds. For example, a compound that modulates PGK1 activity may be administered as a single compound or in combination with another compound that modulates PGK1 activity or that has a different pharmacological activity.

As indicated above, pharmaceutically acceptable salts of the compounds are contemplated and also may be utilized in the disclosed methods. The term “pharmaceutically acceptable salt” as used herein, refers to salts of the compounds which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, alpha-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

The particular counter-ion forming a part of any salt of a compound disclosed herein is may not be critical to the activity of the compound, so long as the salt as a whole is pharmacologically acceptable and as long as the counterion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

In some embodiments, the disclosed therapeutic agents are formulated as time-release preparations. Suitable time-release preparations may include preparations that include a coating that is dissolved in physiological conditions over time or other time-release preparations.

The pharmaceutical compositions may be utilized in methods of treating a neurodegenerative disease or disorder associated with PGK1 activity. For example, the pharmaceutical compositions may be utilized to treat patients having or at risk for acquiring Parkinson's disease (PD), Alzheimer's disease (AD), Huntington's disease (HD), amyotrophic lateral sclerosis (ALS), and Lewy body dementia. Suitable patients include, for example mammals, such as humans and non-human primates (e.g., chimps) or other mammals (e.g., dogs, cats, horses, rats, and mice). Suitable human patients may include, for example, those who have previously been determined to be at risk of having or developing a neurodegenerative disease or disorder associated with PGK1 activity, for example, such as but not limited to PD, AD, HD, ALS, or Lewy body dementia.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

As used herein the term “effective amount” refers to the amount or dose of the compound, upon single or multiple dose administration to the subject, which provides the desired effect in the subject under diagnosis or treatment. The disclosed methods may include administering an effective amount of the disclosed compounds (e.g., as present in a pharmaceutical composition) for treating a disease or disorder associated with PGK1 activity.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

In some embodiments, a typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment.

In some embodiments, compositions can be formulated in a unit dosage form, each dosage containing from about 0.1 to about 500 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

Oral administration is an illustrative route of administering the compounds employed in the compositions and methods disclosed herein. Other illustrative routes of administration include transdermal, percutaneous, intravenous, intramuscular, intranasal, buccal, intrathecal, intracerebral, or intrarectal routes. The route of administration may be varied in any way, limited by the physical properties of the compounds being employed and the convenience of the subject and the caregiver.

As one skilled in the art will appreciate, suitable formulations include those that are suitable for more than one route of administration. For example, the formulation can be one that is suitable for both intrathecal and intracerebral administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, buccal, and/or intrathecal administration but not suitable for intracerebral administration.

The inert ingredients and manner of formulation of the pharmaceutical compositions are conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches, and suspensions. In general, compositions contain from about 0.5% to about 50% of the compound in total, depending on the desired doses and the type of composition to be used. The amount of the compound, however, is best defined as the “effective amount”, that is, the amount of the compound which provides the desired dose to the patient in need of such treatment. The activity of the compounds employed in the compositions and methods disclosed herein are not believed to depend greatly on the nature of the composition, and, therefore, the compositions can be chosen and formulated primarily or solely for convenience and economy.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders.

Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects.

A lubricant can be used in the tablet formulation to prevent the tablet and punches from sticking in the die. The lubricant can be chosen from such slippery solids as talc, magnesium and calcium stearate, stearic acid, and hydrogenated vegetable oils.

Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

When it is desired to administer the compound as a suppository, conventional bases can be used. Illustratively, cocoa butter is a traditional suppository base. The cocoa butter can be modified by addition of waxes to raise its melting point slightly. Water-miscible suppository bases, such as polyethylene glycols of various molecular weights, can also be used in suppository formulations.

Transdermal patches can also be used to deliver the compounds. Transdermal patches can include a resinous composition in which the compound will dissolve or partially dissolve; and a film which protects the composition and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

### EXAMPLES

The followings Example is illustrative only and are not intended to limit the scope of the claimed subject matter.

### Example 1—Enhancing Glycolysis Attenuates Parkinson's Disease Progression in Models and Clinical Databases

Reference is made to Cai et al., “Enhancing glycoylysis attenuates Parkinson's disease progression in models and clinical databases,” J. Clin. Invest. 2019, pages 1-11 and supplemental data, published Sep. 16, 2019, the content of which is incorporated herein by reference in its entirety.

Abstract

Parkinson's disease is a common neurodegenerative disease that lacks therapies to prevent progressive neurodegeneration. Impaired energy metabolism and reduced ATP levels are common features of Parkinson's disease. Previous studies revealed that terazosin enhances the activity of phosphoglycerate kinase 1 (PGK1), thereby stimulating glycolysis and increasing cellular ATP levels. Therefore, we asked if enhancing PGK1 activity would change the course of Parkinson's disease. In toxin-induced and genetic Parkinson's disease models in mice, rats, flies, and induced pluripotent stem cells, terazosin increased brain ATP levels and slowed or prevented neuron loss. It increased dopamine levels and partially restored motor function. Because terazosin is prescribed clinically, we also interrogated two distinct human databases. We found slower disease progression, decreased Parkinson's-related complications, and a reduced frequency of Parkinson's disease diagnoses in people using terazosin and related drugs. These findings suggest that enhancing PGK1 activity and increasing glycolysis may slow neurodegeneration in Parkinson's disease.

Introduction

Parkinson's disease is the second most common neurodegenerative disease. It is estimated to affect ˜6 million people worldwide, and its prevalence will increase further as populations age (1). Patients with PD suffer debilitating motor symptoms as well as non-motor symptoms including dementia and neuropsychiatric abnormalities (2, 3). Dopamine neurons in the substantia nigra pars compacta (SNc) and their projections in the striatum are especially susceptible to disruption in PD (4). Loss and impaired function of dopamine neurons cause the motor abnormalities that are a hallmark feature of PD. Although current treatments can sometimes relieve PD symptoms, no therapies prevent the neurodegeneration (5).

PD may have a number of different causes, and several pathogenic mechanisms have been proposed to contribute to the apoptotic death of neurons (6-10). In the majority of cases, the etiologies are unknown and likely complex. Aging, environmental toxins, and genetic mutations are all risk factors. In many cases, energy deficits and decreased ATP levels are observed in PD (11). First, aging, the major risk factor for PD, impairs cerebral glucose metabolism, reduces mitochondrial biogenesis, and decreases ATP levels (12). Second, glycolysis and mitochondrial function are decreased in people with PD (13, 14). Third, mitochondrial toxins (MPTP, rotenone, paraquat) induce PD and PD-like phenotypes in cells and animals, including humans (15). Fourth, mutations associated with familial PD (e.g., PINK1, LRRK2, α-synuclein, Parkin, DJ-1, CHCHD2) disrupt various aspects of energy metabolism (16). It is also hypothesized that SNc dopaminergic neurons may be particularly susceptible to PD neurodegeneration because their highly branched, unmyelinated axonal arbor, their many neurotransmitter release sites, and their rhythmic firing engender a large metabolic burden (17). These considerations suggested that impaired bioenergetics and reduced ATP levels might contribute to the pathogenesis of PD, might modify the risk of developing PD in the face of PD risk factors, and/or might modify the course or severity of the disease.

We recently discovered that terazosin (TZ) binds and activates phosphoglycerate kinase 1 (PGK1) (18), the first ATP-generating enzyme in glycolysis (FIG. 1A). TZ is an α₁-adrenergic receptor antagonist that can relax smooth muscle and is prescribed to treat benign prostatic hyperplasia and rarely, hypertension (19). However, biochemical and functional studies show that the effects of TZ on PGK1 are independent of α₁-adrenergic antagonism (18). The crystal structure of TZ with PGK1 revealed that the 2,4-diamino-6,7-dimethoxyisoquinazoline motif of TZ binds PGK1 adjacent to the ADP/ATP binding site. In cultured cells, TZ enhanced PGK1 activity thereby increasing ATP levels, and it inhibited apoptosis (18).

The impaired energy production in PD together with the ability of TZ to increase PGK1 activity led us to hypothesize that increasing glycolysis in vivo might slow or prevent the apoptotic neurodegeneration of PD. To test this hypothesis, we tested models of PD in flies, mice, rats, and human cells, and we interrogated human databases to learn if TZ altered the course of disease.

Results

TZ Increases Brain ATP Levels In Vivo in Mice.

To determine if TZ would enhance glycolysis in vivo, we administered it to mice. TZ increased levels of pyruvate, the product of glycolysis, in the SNc and striatum, as well as in cortex (FIG. 1B,1C,9A,9B). Increased pyruvate enhances oxidative phosphorylation (20), and consistent with that, TZ increased citrate synthase activity, a marker of mitochondrial activity (FIG. 1D,9C). Correspondingly, ATP levels increased (FIG. 1E,9D). Like previous in vitro data, the dose-response was biphasic; our previous studies suggest that at low but not high concentrations, TZ may enhance ATP release from PGK1 (18).

We also asked if TZ would increase energy production in mice that received 1-methyl-4-phenyl-1,2,3,6-tetrahydropyridine (MPTP); MPTP causes PD in humans and is used to model PD in other animals (21, 22). Seven days after administering MPTP to mice, pyruvate and ATP levels fell (FIG. 1F,1G,10A-C). Administering TZ prevented the fall in pyruvate and ATP levels. Mitochondrial content (assessed by the ratio of mitochondrial to nuclear DNA, and VDAC and PHB1 levels) also fell (FIG. 10D-F). TZ partially prevented the decrease. As previously suggested (23), the increased pyruvate levels may have stimulated mitochondrial biogenesis. It would be difficult to measure ATP specifically in neurons, however we observed similar changes in human neuroblastoma cells (FIG. 11A-11E). These data indicate that TZ activates glycolysis in vivo. Together with measurements of brain TZ levels (FIG. 11F), they also indicate that TZ readily crosses the blood-brain barrier.

Although PGK1 produces ATP, oxidative phosphorylation is likely important in increasing ATP based on the following. a) Pyruvate, the product of glycolysis and major substrate for the citric acid cycle, increases (FIG. 1C,1F,9B,10B,11A). b) Citrate synthase activity, a marker of mitochondrial activity, increases (FIG. 1D,9C,11B). c) The extracellular acidification rate, a measure of glycolysis, and the O₂ consumption rate, a measure of mitochondrial respiration, both increased (FIG. 11D,11E). d) Mitochondrial content was partially maintained after MPTP (FIG. 10D-10F), which may have also contributed to the increased ATP content.

TZ Decreases MPTP-Induced Neurodegeneration in Mice.

MPTP can model aspects of dopamine neuron loss in mice (21). To determine if PGK1 stimulation would slow or prevent MPTP-mediated deficits, we delivered MPTP, administered TZ for the next 7 days, and then assayed on day 7 (FIG. 2A). Because people with PD present after onset of neuron degeneration, we also asked if delayed TZ administration would slow neuron loss and functional decline. Therefore, in some mice, we waited 7 days after delivering MPTP before starting a 7 day course of TZ treatment. We then assayed on day 14 (FIG. 2A).

Over the course of 14 days, MPTP progressively decreased the levels of tyrosine hydroxylase (TH), the rate-limiting enzyme for generating dopamine. MPTP decreased TH levels in the SNc and striatum, reduced the numbers of TH-positive cells in the SNc, and decreased the intensity of TH immunostaining in their projections in the striatum (FIG. 2B-2G,12A,12B). As a result, the dopamine, 3,4-dihydroxyphenylacetic acid (DOPAC), and homovanillic acid (HVA) content of the striatum and SNc fell (FIG. 2H,2I,12C-12F). MPTP also increased the percentage of TH-positive cells that were TUNEL positive, indicating increased apoptosis (FIG. 2J,12G,12H). Beginning TZ treatment at the time we delivered MPTP attenuated all these defects on day 7. When TZ delivery was delayed for 7 days after MPTP, it improved the abnormalities on day 14. Consistent with these biochemical defects, TZ prevented deficits in motor function at day 7, and it improved motor performance on day 14 after delayed administration (FIG. 2K,12I,12J).

These in vivo results in mice suggest that TZ slows or prevents MPTP-induced neurodegeneration, partially restores TH and dopamine levels, and improves motor function.

TZ Enhancement of PGK1 Activity Slows Neurodegeneration in 6-OHDA-Treated Rats.

6-hydroxydopamine (6-OHDA) is delivered to rats as a model of dopamine neuron degeneration in PD (24). Previous studies have shown progressive cell death and injury between 2 and 12 weeks after delivering 6-OHDA (25-27). Therefore, we chose a 7 week course of observation. We injected 6-OHDA into the right striatum, waited 2-5 weeks, and then initiated a two-week course of TZ (FIG. 3A). In vehicle-treated rats, evidence of SNc cell apoptosis progressively increased from 2 to 7 weeks (FIG. 3B,13A,13B). However, irrespective of the delay until beginning treatment, TZ attenuated further cell loss. 6-OHDA also progressively decreased TH levels in the striatum and SNc (FIG. 3C,13C-13F). The percentage of TH-positive cells in the SNc and the intensity of TH immunostaining in the striatum also fell (FIG. 3D,3E). TZ partially reverted these abnormalities toward control values. 6-OHDA progressively decreased the dopamine, DOPAC, and HVA content, and TZ partially prevented the reduction (FIG. 3F,13G,13H). Seven weeks after injecting 6-OHDA into the right striatum, use of the left forepaw had fallen (FIG. 3G). However, when rats received TZ between weeks 5 and 7, they used both forepaws equally.

Previous studies have shown that MPTP and 6-OHDA can rapidly reduce TH expression (21), and consistent with that, TH levels, TH-positive neurons, TH intensity in the striatum, and dopamine content decreased rapidly after MPTP and 6-OHDA administration to mice and rats, respectively (FIG. 2,3,12,13). Cell death was also apparent. However, not all the damaged cells were rapidly killed; cell death continued to progress for at least 14 days in MPTP/mice and 7 weeks in 6-OHDA/rats (FIG. 2J,3B). Accordingly, TH levels, TH-positive neurons, TH intensity in the striatum, and dopamine content also continued to decrease further with time. Administering TZ, even after the onset of neurodegeneration, slowed cell death, and it increased TH levels, dopamine content, and motor performance compared to vehicle-treated controls (FIG. 2,3,12,13).

After MPTP and 6-OHDA administration, apoptotic cell death continued for 14 days and 7 weeks, respectively. Delayed TZ administration (beginning at day 7 in MPTP/mice and week 5 in 6-OHDA/rats) slowed or prevented further apoptotic cell death. In MPTP/mice, dopamine levels, behavioral performance, and in some cases TH levels at day 14 exceeded those at day 7. Likewise, in 6-OHDA/rats, dopamine levels and TH levels at week 7 exceeded those at week 5. PD neurons that have not yet undergone apoptotic cell death almost certainly have impaired metabolic function (28). Our results suggest that TZ improved the function of neurons that were impaired by MPTP and 6-OHDA, but had not yet degenerated.

TZ Enhances PGK Activity to Attenuate Rotenone-Induced Neurodegeneration in Flies.

As an additional model of PD, we treated Drosophila melanogaster with rotenone, a mitochondrial complex I inhibitor implicated in sporadic PD (29). Rotenone exposure reduced brain ATP levels (FIG. 4A,4B). It also disrupted motor function assayed by climbing behavior (FIG. 4C). PGK is highly conserved in flies and mammals, and supplying TZ together with rotenone minimized decrements in ATP content and motor performance.

Previous studies showed that TZ increases ATP by enhancing PGK1 activity (18, 30). We knocked-down Pgk in Drosophila by expressing RNAi and found that it abolished the protective effect of TZ on motor performance (FIG. 4D,4E vs. 4C). Conversely, overexpressing PGK1 in Drosophila TH neurons, all neurons (Appl promoter), or all cells (actin promoter) made flies resistant to rotenone-induced behavioral defects (FIG. 4F). In contrast, expression in muscle was not protective. These results together with earlier findings (18, 30) indicate that TZ protects TH neurons by activating PGK1.

TZ Attenuates Neurodegeneration in Genetic Models of PD.

In addition to toxin-induced models, we tested fly, mouse, and human genetic models of PD. PINK1 mutations cause PD in humans; we therefore tested the Drosophila PINK1⁵ mutant (31-33). We administered vehicle or TZ from day 1 after hatching to day 10. On day 10, nearly all PINK1⁵ flies exhibited wing posture defects (FIG. 5A). TZ partially reversed this abnormality. Brain TH and ATP levels also decreased, and motor performance was impaired in PINK1⁵ flies (FIG. 5B-5E,14). TZ partially corrected these defects. We also tested the Drosophila LRRK^{ex1} mutant (34); LRRK2 mutations cause autosomal-dominant, late-onset PD (35). TZ also attenuated motor deficits in that model (FIG. 5F).

Abnormal accumulation of α-synuclein, a major constituent of Lewy bodies, is a key feature of PD (36). Transgenic mice overexpressing wild-type human α-synuclein (mThyl-hSNCA) exhibit PD-like neurodegeneration at an advanced age (37). When they were 3 months-old, we began treating mThyl-hSNCA mice with vehicle or TZ. When they were 15 months old, vehicle-treated mice had substantial human α-synuclein in the striatum and SNc (FIG. 5G-J) and impaired motor performance on the rotarod and pole test (FIG. 5K,15). TZ treatment partially prevented these abnormalities.

We also tested the effect of TZ on dopamine neurons differentiated from induced pluripotent stem cells (iPSCs). LRRK2^{G2019S} is the most common LRRK2 mutation and is associated with ˜4% of familial and ˜1% of sporadic PD (38). Dopamine neurons derived from LRRK2^{G2019S} iPSCs recapitulate PD features including abnormal α-synuclein accumulation (39). We studied such neurons generated from two patients. After thirty days of differentiation, the dopamine neurons showed no overt signs of neurodegeneration (FIG. 16). However, ˜60% of the LRRK2^{G2019S} dopamine neurons had accumulated α-synuclein compared to ˜15% of dopamine neurons from healthy individuals (FIG. 6A,6B). Adding TZ for 24 hr increased the ATP content and reduced the percentage of LRRK2^{G2019S} dopamine neurons with elevated a-synuclein accumulation (FIG. 6A-6C).

In the PPMI Database, People with PD Who were Taking TZ had a Reduced Rate of Progressive Motor Disability.

In the past, assessing whether an agent might affect PD has been largely limited to animal models. Three factors allowed us to assess efficacy in humans. First, TZ is a relatively commonly used drug. Second, availability of human clinical databases allowed us to test for a TZ effect. Third, tamsulosin can serve as a control for TZ. Like TZ, tamsulosin is an α1-adrenergic antagonist, and like TZ, tamsulosin is prescribed for benign prostatic hyperplasia. However in contrast to TZ, tamsulosin does not have a quinazoline motif that binds to and enhances PGK1 activity.

PD is common in older men; PD incidence increases markedly after age 60, and the prevalence in men is approximately 1.5 times that in women (40). TZ is prescribed for benign prostatic hyperplasia, a disease that also affects older men. Therefore, we suspected that some patients with PD used TZ, and we hypothesized that they would have a reduced rate of disease progression. To test this hypothesis, we interrogated the Parkinson's Progression Markers Initiative (PPMI) database. This database enrolls patients with PD shortly after diagnosis and follows their motor function as assayed by the Movement Disorder Society's Unified Parkinson's Disease Rating Scale Part 3 (41). Although this clinical database is small, it is relatively unique in assessing motor progression. We identified 7 men with PD who used TZ and compared them to 269 men not taking TZ. Compared to the controls, patients who used TZ had a slower rate of motor function decline (Table 1).

TABLE 1
Subjects from the PPMI Database

| | Controls | Tamsulosin | TZ | TZ/DZ/AZ |
|---|---|---|---|---|
| Number of PD patients | 269 | 24 | 7 | 13 |
| Dosage mg/d | NA | 0.4 ± 0.1 | 5.0 ± 2.5 | TZ 5.0 ± 2.5 |
| Mean (SD) | | | | DZ 3.3 ± 1.2 |
| | | | | AZ 10.0 ± 0.0 |
| MDS-UPDRS Part 3 | 20.2 ± 9.4 | 20.3 ± 7.1 | 19.1 ± 12.8 | 20.9 ± 12.2 |
| Baseline score, | | | | |
| mean ± S.D. | | | | |
| P (vs. controls) | NA | 0.961 | 0.771 | 0.81 |
| MDS-UPDRS Part 3 | 0.54 ± 0.05 | 0.39 ± 0.14 | 0.01 ± 0.25 | 0.02 ± 0.20 |
| Slope of change/month, | | | | |
| mean ± S.E. | | | | |
| P (vs. controls) | NA | 0.301 | 0.038 | 0.013 |

*TZ/DZ/AZ includes PD patients taking TZ (n = 7), doxazosin (n = 3), or altuzosin (n = 3). When comparing TZ to controls, the 6 participants taking doxazosin and altuzosin were removed (as opposed to being considered part of the Controls group). Statistical analysis was linear mixed effects regression and is further described in the supplementary material. MDS-UPDRS scores were obtained when the participants were not yet taking a PD medication or were in the practically defined OFF state (at least 6 hours after the last dose of levodopa or any other anti-PD medication).

Although the difference was statistically significant, only 7 patients used TZ. We therefore sought a larger sample.

The crystal structure of TZ with PGK1 (18) suggested that related drugs with quinazoline motifs might also enhance PGK1 activity. Consistent with that possibility doxazosin and alfuzosin increased glycolysis in M17 cells and tyrosine hydroxylase levels in MPTP-treated mice (FIG. 17A,17B). We identified 13 men in the PPMI database using TZ, doxazosin, or alfuzosin (TZ/DZ/AZ) (Table 1). Progression of motor disability was slowed in those patients (FIG. 7, 18, Table 1).

In contrast to TZ, doxazosin, and alfuzosin, tamsulosin lacks a quinazoline motif for binding to PGK1. Consistent with that, tamsulosin did not rescue tyrosine hydroxylase levels in MPTP-treated mice (FIG. 17B). Correspondingly, tamsulosin failed to slow the motor function decline of patients enrolled in the PPMI database (FIG. 7, Table 1). These data are also consistent with the conclusion that enhanced glycolytic activity and attenuation of cell death are mediated by TZ's effect on PGK1 and not α1-adrenergic receptors.

The IBM Watson/Truven Database Shows that People with PD Who Used TZ/DZ/AZ had Fewer PD-Related Diagnoses.

To evaluate a larger number of people with PD and to use a different database and assessment methods, we interrogated the IBM Watson/Truven Health Analytics MarketScan Database from 2011 to 2016. The database includes longitudinal, de-identified diagnoses (ICD-9/ICD-10 codes) and pharmaceutical claims. We identified 2,880 PD patients taking TZ/DZ/AZ (4,821 person years) (Table 2).

TABLE 2
Subjects from the Truven Marketscan database.

| | Tamsulosin | TZ | Doxazosin | Alfuzosin | TZ/DZ/AZ |
|---|---|---|---|---|---|
| Number of enrollees | 15,409 | 1,173 | 1,177 | 529 | 2,880 |
| Person-years of exposure | 21,409 | 2,046 | 1,967 | 808 | 4,821 |
| Dosage mg/d | 0.4 ± 0.0 | 4.6 ± 3.1 | 3.9 ± 2.3 | 10 | NA |
| Mean ± SD | | | | | |
| Age | 77.2 ± 7.7 | 77.8 ± 7.4 | 77.6 ± 7.7 | 75.9 ± 8.0 | 77.4 ± 7.7 |
| Mean ± SD | | | | | |

*Summary of the number of enrollees, duration of exposure and dose of drugs. Age refers to the age of the patient at the first observed medication dispensing event. The first event can be the age of a patient at the time of a refill of a prescription that was begun prior to entry of the patient into the Truven database, or it can be the age of a patient who began the medication during the Truven observation period.

For a comparison group, we chose PD patients taking tamsulosin; that controls for use of an α1-adrenergic antagonist and for the presence of benign prostatic hyperplasia. We identified 15,409 individuals with PD taking tamsulosin (21,409 person years). To obtain a list of diagnostic codes associated with PD, we first identified the 497 most common diagnostic codes in the group of people with PD. Then, two neurologists who care for patients with PD identified 79 potentially PD-related diagnoses (data not shown).

Using a quasi-Poisson generalized linear model, we found that the relative risk of having any of the 79 PD-related diagnostic codes was 0.78 (95% CI:0.74-0.82) for the TZ/DZ/AZ group relative to those on tamsulosin (P<0.00001). Of the 79 PD-related codes, we found a reduced risk in 69 codes among PD patients taking TZ/DZ/AZ vs. those taking tamsulosin (FIG. 8A). Moreover 41 diagnostic codes were statistically significantly decreased in the PD patients taking TZ/DZ/AZ vs. those taking tamsulosin, whereas only 2 diagnostic codes were significantly increased in the TZ/DZ/AZ group.

To estimate PD-related benefits/risks attributable to TZ/DZ/AZ vs. tamsulosin, we calculated the relative risk (RR) for clinically-relevant groupings of the 79 PD-related codes. Relative to PD patients taking tamsulosin, those on TZ/DZ/AZ had reduced clinic/hospital encounters for motor symptoms (RR 0.77 (95% CI:0.70-0.84)), non-motor symptoms (RR 0.78 (95% CI:0.73-0.83)), and PD complications (RR 0.76 (95% CI:0.71-0.82)) (FIG. 8B, and data not shown). Of note, dopamine analogs do not treat PD symptoms such as dementia and neuropsychiatric manifestations (3). However, the RR for these diagnostic codes were also less than 1.0.

These data suggest that under real-world conditions, TZ and related drugs that enhance PGK1 activity reduce PD signs, symptoms and complications.

People Who Used TZ/DZ/AZ had a Decreased Risk of PD Diagnoses.

We also used the Truven database to test whether TZ/DZ/AZ might reduce the frequency of PD diagnoses. We identified 78,444 PD-free enrollees who were taking TZ/DZ/AZ. During a follow-up duration of 284±382 days (mean±SD) a total of 118 people (0.15%) developed PD. In contrast, in an equal-sized cohort of PD-free enrollees taking tamsulosin and matched on age and follow-up duration (284±381 days), 190 people (0.25%) developed PD. The hazard ratio from the Cox proportional hazards regression for the matched cohort was 0.62 (95% CI: 0.49-0.78) (P<0.0001).

Discussion

Our results indicate that in both toxin-induced and genetic models of PD in multiple animal species, enhancing PGK1 activity slows or prevents neurodegeneration in vivo, thereby increasing dopamine levels and improving motor performance. Enhancing PGK1 activity showed beneficial effects, even when begun after the onset of neurodegeneration. Moreover, interrogating two independent databases suggested that TZ and related quinazoline agents slowed disease progression, reduced PD-related complications in people with PD, and reduced the risk of receiving a PD diagnosis.

Evidence from several experiments indicates that TZ elicits its beneficial effects in PD by enhancing the activity of PGK1 and not by inhibiting the α₁-adrenergic receptor. a) Our earlier experiments and crystal structure show that the quinazoline motif of TZ binds PGK1 near the nucleotide binding site (18). b) Studies with recombinant PGK1, studies with cultured cells, and measurements in brain following in vivo delivery all reveal a biphasic relationship between the concentration of TZ and ATP levels (18). c) Tamsulosin inhibits α₁-adrenergic receptors, but its structure lacks a quinazoline group that binds PGK1, it does not enhance glycolysis, and it does not prevent the reduction of tyrosine hydroxylase levels in MPTP-treated mice. In contrast, two drugs that have a structure similar to TZ (doxazosin and alfuzosin) enhance glycolysis in vitro and protect MPTP-treated mice. d) Knocking-down Pgk1 in Drosophila TH-neurons abolished the protective effect of TZ. e) Overexpressing PGK1 in flies, mice, and fish phenocopied effects of TZ (18, 30). f) TZ was active in Drosophila melanogaster, which do not have α₁-adrenergic receptors. Allosteric and covalent regulatory mechanisms have been identified for most glycolytic enzymes. For example, insulin-stimulated deacetylation increases PGK1 activity, and disrupting that regulation results in glycolytic insufficiency (42).

Previous work has identified numerous genetic mutations and several environmental factors that cause or predispose to PD (11-16, 43). As indicated above, reduced energy metabolism and decreased ATP levels are a feature of many of these environmental and genetic factors, as is aging, the major PD risk factor. Therefore, enhancing glycolysis might slow progression in PD of several etiologies.

This study does not reveal how enhanced glycolysis slows neurodegeneration and progression in PD. However, the increased ATP levels produced by TZ may be key. ATP has properties of a hydrotrope; it can prevent aggregate formation and dissolve previously formed protein aggregates (44, 45). Moreover, the transition between aggregate stability and dissolution occurs in a narrow range at physiological ATP concentrations. We speculate that by elevating ATP levels, TZ facilitates solubilization of aggregates, including α-synuclein, and prevents the neurodegeneration of PD. However, other mechanisms are also possible including ATP-dependent disaggregases and chaperones (such as HSP90) that reduce apoptosis (18, 44, 45).

This study also has limitations. First, toxin-induced and genetic models of PD have limitations (46). Toxins such as MPTP and rotenone can cause PD in humans and PD-like disease in animals. Genetic defects also cause PD in humans and PD-like disease in animals. However, most PD is age-related with etiologies that remain unidentified and are likely complex. Moreover, no current model unequivocally or accurately predicts therapeutic benefit or pathogenesis. It is exactly for these reasons that we used multiple animal models of PD and that we sought out human data. Second, our analysis of human databases was limited to men, because they are the ones who are treated for benign prostatic hyperplasia. However, we expect that similar results would be obtained in women. Third, our data from humans are retrospective. However, the human data provide compelling evidence that cannot be obtained from animal models alone. Fourth, our analysis of the PPMI and Truven databases compared patients on TZ/DZ/AZ to those on tamsulosin. Although all the drugs were prescribed for benign prostatic hyperplasia, we cannot exclude that some other factor might have influenced prescribing behavior. For example, orthostatic hypotension is a complication of both the autonomic dysfunction in PD and of the drugs, and there are reports suggesting that tamsulosin may elicit less orthostatic hypotension than TZ (47). However, such an effect would not explain the PPMI conclusions. Interestingly, the risk of orthostatic hypotension and falls was reduced, not increased, for PD patients taking TZ/DZ/AZ vs. those on tamsulosin. In PD, neurons that have not yet degenerated almost certainly have compromised cellular function (28), and we speculate that TZ/DZ/AZ improved their functional integrity.

Results from this study together with earlier data lead us to three additional speculations. First, TZ is already used clinically, and in this regard, it is interesting that several studies reported that TZ improved glucose metabolism in diabetic patients (48, 49). That observation has gone unexplained. We speculate that stimulation of PGK1 activity might have been responsible. Consistent with that conjecture and with the conclusion that an α₁-adrenergic receptor antagonistic effect was not responsible, α₁-adrenergic receptor antagonists structurally unrelated to TZ lacked that effect. In addition, disrupting the α_{1B}-adrenergic receptor in mice had an effect opposite of TZ (50). Second, loss of function PGK1 mutations cause recessive hemolytic anemia, myopathy, seizures, and intellectual disability. However, Parkinsonism has also been reported (51, 52). The authors speculated that reduced ATP generation in the SNc may have been responsible. Third, PD occurs ˜1.5 times more frequently in men than women (53). Why males are predisposed is unknown. However, it may be worth noting that the PGK1 gene is located on the X-chromosome. Thus, the consequences of DNA sequence variations that could subtly reduce PGK1 levels or activity might more likely manifest in men than women.

Finding that TZ increases glycolysis and prevents progressive neurodegeneration suggests that energy deficits might either be a pathogenic factor in the pathogenesis of PD or predispose to PD in the presence of environmental or genetic etiologies (11, 16). These findings identify a protein and a pathway that might be targeted to slow or prevent neurodegeneration in PD and potentially other neurodegenerative diseases with altered energy balance (54).

Methods

The supplemental data contain information on the materials, reagents, experimental procedures, and analysis methods.

Statistical and Analysis Considerations.

For experiments to quantify animal behavior and for sample collections, experimenters were blinded to genotype and intervention, and studies were done by two different experimenters. Numbers of animals studied were based on our past experience and preliminary data. In all figures, data points are from individual mice and rats, or groups of flies. We did not exclude any data points from this study. Bars and whiskers indicate mean±SEM. Blue indicates controls and red indicates TZ treatment. Statistical significance for comparisons between data sets was primarily with non-parametric tests. For studies of fly motor performance, our previous studies showed that within a group of flies (15-50 flies for one data point), the data fit a gaussian distribution. Moreover, multiple groups of flies also fit a gaussian distribution. Therefore, parametric tests were used to evaluate statistical significance in studies using flies, and ANOVA evaluations were 1-way. All statistical tests were two-tailed. A p value <0.05 was considered statistically significant. On individual graphs, we show statistical significance for the main comparisons with asterisks *p<0.05, **p<0.01, ***p<0.001. Table S5 shows the statistical tests used for all data and the resulting p values for comparisons.

Study Approval.

All experiments using mice and rats were approved by the Institutional Animal Care and Use Committee, Peking University, Beijing (Approval NO: LSC-Liu1-1 and LSC-Liu1-2).

### REFERENCES

1. G.B.D.NeurologyCollaborators. Global, regional, and national burden of neurological disorders, 1990-2016: a systematic analysis for the Global Burden of Disease Study 2016. Lancet Neurol. 2019; 18(5):459-80.
2. Fahn S. The history of dopamine and levodopa in the treatment of Parkinson's disease. Mov Disord. 2008; 23 Suppl 3:S497-508.
3. Chaudhuri K R, and Odin P. The challenge of non-motor symptoms in Parkinson's disease. Prog Brain Res. 2010; 184:325-41.
4. Kalia L V, and Lang A E. Parkinson's disease. Lancet. 2015; 386(9996):896-912.
5. Maiti P, Manna J, and Dunbar G L. Current understanding of the molecular mechanisms in Parkinson's disease: Targets for potential treatments. Transl Neurodegener. 2017; 6:28.
6. Braak H, and Del Tredici K. Neuropathological Staging of Brain Pathology in Sporadic Parkinson's disease: Separating the Wheat from the Chaff. J Parkinsons Dis. 2017; 7(s1):571-585.
7. Hunn B H, Cragg S J, Bolam J P, Spillantini M G, and Wade-Martins R. Impaired intracellular trafficking defines early Parkinson's disease. Trends Neurosci. 2015; 38(3):178-88.
8. Johnson M E, Stecher B, Labrie V, Brundin L, and Brundin P. Triggers, Facilitators, and Aggravators: Redefining Parkinson's Disease Pathogenesis. Trends Neurosci. 2019; 42(1):4-13.
9. Lunati A, Lesage S, and Brice A. The genetic landscape of Parkinson's disease. Rev Neurol (Paris). 2018; 174(9):628-43.
10. Grunewald A, Kumar K R, and Sue C M. New insights into the complex role of mitochondria in Parkinson's disease. Prog Neurobiol. 2018.
11. Saxena U. Bioenergetics failure in neurodegenerative diseases: back to the future. Expert Opin Ther Targets. 2012; 16(4):351-4.
12. Hoyer S. Brain glucose and energy metabolism during normal aging. Aging (Milano). 1990; 2(3):245-58.
13. Firbank M J, Yarnall A J, Lawson R A, Duncan G W, Khoo T K, Petrides G S, et al. Cerebral glucose metabolism and cognition in newly diagnosed Parkinson's disease: ICICLE-P D study. J Neurol Neurosurg Psychiatry. 2017; 88(4):310-6.
14. Schapira A H, Cooper J M, Dexter D, Clark J B, Jenner P, and Marsden C D. Mitochondrial complex I deficiency in Parkinson's disease. J Neurochem. 1990; 54(3):823-7.
15. Blesa J, Phani S, Jackson-Lewis V, and Przedborski S. Classic and new animal models of Parkinson's disease. J Biomed Biotechnol. 2012; 2012:845618.
16. Schapira A H. Mitochondria in the aetiology and pathogenesis of Parkinson's disease. Lancet Neurol. 2008; 7(1):97-109.
17. Surmeier D J. Determinants of dopaminergic neuron loss in Parkinson's disease. FEBS J. 2018; 285(19):3657-68.
18. Chen X, Zhao C, Li X, Wang T, Li Y, Cao C, et al. Terazosin activates Pgk1 and Hsp90 to promote stress resistance. Nat Chem Biol. 2015; 11(1):19-25.
19. Wilt T J, Howe W, and MacDonald R. Terazosin for treating symptomatic benign prostatic obstruction: a systematic review of efficacy and adverse effects. British Journal of Urology. 2002; 89(3):214-25.
20. Compan V, Pierredon S, Vanderperre B, Krznar P, Marchiq I, Zamboni N, et al. Monitoring Mitochondrial Pyruvate Carrier Activity in Real Time Using a BRET-Based Biosensor: Investigation of the Warburg Effect. Mol Cell. 2015; 59(3):491-501.
21. Heikkila R E, Hess A, and Duvoisin R C. Dopaminergic neurotoxicity of 1-methyl-4-phenyl-1,2,5,6-tetrahydropyridine in mice. Science. 1984; 224(4656):1451-3.
22. Przedborski S, Jackson-Lewis V, Djaldetti R, Liberatore G, Vila M, Vukosavic S, et al. The parkinsonian toxin MPTP: action and mechanism. Restor Neurol Neurosci. 2000; 16(2): 135-42.
23. Wilson L, Yang Q, Szustakowski J D, Gullicksen P S, and Halse R. Pyruvate induces mitochondrial biogenesis by a PGC-1 alpha-independent mechanism. Am J Physiol Cell Physiol. 2007; 292(5):C1599-605.
24. Ungerstedt U. 6-Hydroxy-dopamine induced degeneration of central monoamine neurons. Eur J Pharmacol. 1968; 5(1):107-10.
25. He Y, Lee T, and Leong S K. Time course of dopaminergic cell death and changes in iron, ferritin and transferrin levels in the rat substantia nigra after 6-hydroxydopamine (6-OHDA) lesioning. Free Radic Res. 1999; 31(2):103-12.
26. Harms A S, Barnum C J, Ruhn K A, Varghese S, Trevino I, Blesch A, et al. Delayed dominant-negative TNF gene therapy halts progressive loss of nigral dopaminergic neurons in a rat model of Parkinson's disease. Mol Ther. 2011; 19(1):46-52.
27. Yuan W J, Yasuhara T, Shingo T, Muraoka K, Agari T, Kameda M, et al. Neuroprotective effects of edaravone-administration on 6-OHDA-treated dopaminergic neurons. BMC Neurosci. 2008; 9:75.
28. Braak H, and Del Tredici K. Potential Pathways of Abnormal Tau and alpha-Synuclein Dissemination in Sporadic Alzheimer's and Parkinson's Diseases. Cold Spring Harb Perspect Biol. 2016; 8(11): 1-23.
29. Coulom H, and Birman S. Chronic exposure to rotenone models sporadic Parkinson's disease in Drosophila melanogaster. J Neurosci. 2004; 24(48):10993-8.
30. Boyd P J, Tu W Y, Shorrock H K, Groen E J N, Carter R N, Powis R A, et al.

Bioenergetic status modulates motor neuron vulnerability and pathogenesis in a zebrafish model of spinal muscular atrophy. PLoS Genet. 2017; 13(4):e1006744.
31. Clark I E, Dodson M W, Jiang C, Cao J H, Huh J R, Seol J H, et al. Drosophila pink1 is required for mitochondrial function and interacts genetically with parkin. Nature. 2006; 441(7097):1162-6.
32. Tsai P I, Lin C H, Hsieh C H, Papakyrikos A M, Kim M J, Napolioni V, et al. PINK1 Phosphorylates MIC60/Mitofilin to Control Structural Plasticity of Mitochondrial Crista Junctions. Mol Cell. 2018; 69(5):744-56 e6.
33. Yun J, Cao J H, Dodson M W, Clark I E, Kapahi P, Chowdhury R B, et al. Loss-of-function analysis suggests that Omi/HtrA2 is not an essential component of the PINK1/PARKIN pathway in vivo. J Neurosci. 2008; 28(53):14500-10.
34. Lee S B, Kim W, Lee S, and Chung J. Loss of LRRK2/PARK8 induces degeneration of dopaminergic neurons in Drosophila. Biochem Biophys Res Commun. 2007; 358(2):534-9.
35. Zimprich A, Biskup S, Leitner P, Lichtner P, Farrer M, Lincoln S, et al. Mutations in LRRK2 cause autosomal-dominant parkinsonism with pleomorphic pathology. Neuron. 2004; 44(4):601-7.
36. Spillantini M G, Crowther R A, Jakes R, Hasegawa M, and Goedert M. alpha-Synuclein in filamentous inclusions of Lewy bodies from Parkinson's disease and dementia with lewy bodies. Proc Natl Acad Sci USA. 1998; 95(11):6469-73.
37. Rockenstein E, Mallory M, Hashimoto M, Song D, Shults C W, Lang I, et al. Differential neuropathological alterations in transgenic mice expressing alpha-synuclein from the platelet-derived growth factor and Thy-1 promoters. J Neurosci Res. 2002; 68(5):568-78.
38. Lill C M. Genetics of Parkinson's disease. Mol Cell Probes. 2016; 30(6):386-96.
39. Nguyen H N, Byers B, Cord B, Shcheglovitov A, Byrne J, Gujar P, et al. LRRK2 mutant iPSC-derived D A neurons demonstrate increased susceptibility to oxidative stress. Cell Stem Cell. 2011; 8(3):267-80.
40. Driver J A, Logroscino G, Gaziano J M, and Kurth T. Incidence and remaining lifetime risk of Parkinson disease in advanced age. Neurology. 2009; 72(5):432-8.
41. Goetz C G, Tilley B C, Shaftman S R, Stebbins G T, Fahn S, Martinez-Martin P, et al. Movement Disorder Society-sponsored revision of the Unified Parkinson's Disease Rating Scale (MDS-UPDRS): scale presentation and clinimetric testing results. Mov Disord. 2008; 23(15):2129-70.
42. Wang S, Jiang B, Zhang T, Liu L, Wang Y, Wang Y, et al. Insulin and mTOR Pathway Regulate HDAC3-Mediated Deacetylation and Activation of PGK1. PLoS Biol. 2015; 13(9):e1002243.
43. Zanon A, Pramstaller P P, Hicks A A, and Pichler I. Environmental and Genetic Variables Influencing Mitochondrial Health and Parkinson's Disease Penetrance. Parkinsons Dis. 2018; 2018:8684906.
44. Patel A, Malinovska L, Saha S, Wang J, Alberti S, Krishnan Y, et al. ATP as a biological hydrotrope. Science. 2017; 356(6339):753-6.
45. Hayes M R, Peuchen E H, Dovichi N J, and Weeks D L. Dual roles for ATP in the regulation of phase separated protein aggregates in Xenopus oocyte nucleoli. Elife. 2018; 7.
46. Dawson T M, Golde T E, and Lagier-Tourenne C. Animal models of neurodegenerative diseases. Nat Neurosci. 2018; 21(10):1370-9.
47. Dong Z, Wang Z, Yang K, Liu Y, Gao W, and Chen W. Tamsulosin versus terazosin for benign prostatic hyperplasia: a systematic review. Syst Biol Reprod Med. 2009; 55(4): 129-36.
48. Kirk J K, Konen J C, Shihabi Z, Rocco M V, and Summerson J H. Effects of Terazosin on Glycemic Control, Cholesterol, and Microalbuminuria in Patients with Non-Insulin-Dependent Diabetes Mellitus and Hypertension. Am J Ther. 1996; 3(9):616-21.
49. Shionoiri H, Gotoh E, Ito T, Hata T, Iwatsubo H, Takegawa K, et al. Long-term therapy with terazosin may improve glucose and lipid metabolism in hypertensives: a multicenter prospective study. Am J Med Sci. 1994; 307 Suppl 1:S91-5.
50. Boyda H N, Procyshyn R M, Pang C C, and Barr A M. Peripheral adrenoceptors: the impetus behind glucose dysregulation and insulin resistance. J Neuroendocrinol. 2013; 25(3):217-28.
51. Sakaue S, Kasai T, Mizuta I, Suematsu M, Osone S, Azuma Y, et al. Early-onset parkinsonism in a pedigree with phosphoglycerate kinase deficiency and a heterozygous carrier: do PGK-1 mutations contribute to vulnerability to parkinsonism? NPJ Parkinsons Dis. 2017; 3:13.
52. Sotiriou E, Greene P, Krishna S, Hirano M, and DiMauro S. Myopathy and parkinsonism in phosphoglycerate kinase deficiency. Muscle Nerve. 2010; 41(5):707-10.
53. Wooten G F, Currie L J, Bovbjerg V E, Lee J K, and Patrie J. Are men at greater risk for Parkinson's disease than women? J Neurol Neurosurg Psychiatry. 2004; 75(4):637-9.
54. Yao J, Irwin R W, Zhao L, Nilsen J, Hamilton R T, and Brinton R D. Mitochondrial bioenergetic deficit precedes Alzheimer's pathology in female mouse model of Alzheimer's disease. Proc Natl Acad Sci USA. 2009; 106(34):14670-5.

Supplemental Methods

Chemicals.

1-Methyl-4-phenyl-1,2,3,6-tetrahydropyridine (MPTP), 6-Hydroxydopamine hydrobromide (6-OHDA), 1-methyl-4-phenylpyridinium (MPP⁺), rotenone, apomorphine hydrochloride, tamsulosin hydrochloride, urapidil hydrochloride, phenylephrine hydrochloride, terazosin hydrochloride, doxazosin mesylate, alfuzosin hydrochloride, and prazosin hydrochloride were purchased from Sigma-Aldrich (St. Louis, Mo., USA). The In Situ Cell Death Detection Kit was purchased from Roche Diagnostics (USA). The 3, 3-diaminobenzidine (DAB) kit was purchased from Beijing ComWin Biotech Co. Ltd. (Beijing, China). The EnzyChrom Pyruvate Assay Kit was purchased from BioAssay Systems (Hayward, Calif., USA). The ATP assay kit was purchased from Promega Biotech (Beijing, China). The BCA Protein Assay Kit was purchased from Vigorous Biotechnology Beijing (Beijing, China). The Citrate Synthase (CS) Assay Kit was purchased from Nanjing Jiancheng Bioengineering Institute (Nanjing, China). The Nissl staining kit was purchased from Beyotime Biotech. (Beijing, China).

Antibodies.

The antibodies used in immunohistochemistry and immunofluorescence were as follows: rabbit anti-tyrosine hydroxylase (1:2000, AB152, Millipore), goat anti-rabbit secondary antibody (1:200, BE0101, EasyBio Technology Co., Ltd.), and chicken anti-GFP (1:500, ab13970, Abcam). The antibodies used in iPSCs were mouse anti-human α-synuclein (610787, BD Biosciences, Madrid, Spain), rabbit anti-TH (sc-14007, Santa Cruz Biotechnology, Madrid), and mouse anti-TUJ1 (801202, Biolegend). The antibodies used in western blot were rabbit anti-TH (1:1000, AB152, Millipore), mouse anti-Pgk1/2 (1:200, sc-48342, Santa), mouse anti-human α-synuclein (1:500, 610787, BD Biosciences), rabbit anti-VDAC (1:1000, 8674, Cell Signaling), rabbit anti-PHB1 (1:1000, 8674, Cell Signaling) and mouse anti-β-actin (1:2000, HC201, TransGen Biotech).

Cell Culture and Hypoxia Induction.

The human BE(2)-M17 neuroblastoma cells were purchased from National Experimental Cell Resource Sharing Service Platform (Beijing, China). Cells were cultured in DMEM (Dulbecco's modified Eagle's medium) (Gibco), supplemented with 10% fetal bovine serum (Gibco) and 1% penicillin-streptomycin (Gibco), at 37° C. with 5% CO₂ and 95% air atmosphere in a humidified incubator (Thermo Scientific). Cells were incubated with mild hypoxia for 12 hr before study. Cells were cultured in a sealed chamber (Stemcell Technologies Vancouver, Canada) that was flushed with a humidified gas mixture composed of 5% 02, 5% carbon dioxide (CO₂), and 90% nitrogen (N₂) for 12 hr. Three hours before harvest, the cells were switched to 5% CO₂ and 95% O₂ (1, 2). TZ (10 μM) or vehicle was added to the medium 15 hr before harvest. Assays of ATP, pyruvate, and citrate synthase activities (CS) were performed according to the manufacturer's instructions.

Animal Maintenance.

Male C57BL/6J mice (7 weeks old) and male Sprague-Dawley (SD) rats (200-220 g) were purchased from the Vital River Laboratory Animal Technology (Beijing, China). Animals were housed under a 12 hr light/dark cycle with free access for food and water. All experiments using mice and rats were approved by the Institutional Animal Care and Use Committee, Peking University, Beijing (Approval NO: LSC-Liu1-1 and LSC-Liu1-2).

SNCA Transgenic Mice.

SNCA transgenic mice(mThyl-hSNCA) were purchased from the Jackson Laboratory (017682, line15). mThyl-hSNCA express the human α-synuclein gene under the direction of the mouse thymus cell antigen 1 promoter (3). Mice were treated with TZ (0.03 mg/kg, oral) or vehicle from 3 months old and sacrificed at 15 months old. Behavioral tests were carried out during the TZ treatment period.

MPTP Mouse Model.

After one week housing to adopt to the new environment, mice were randomly divided into six groups including the control group (saline injection) and the TZ group (0.1 μg/kg, 1 μg/kg, 10 μg/kg, 100 μg/kg and 1000 μg/kg). MPTP was injected (i.p.) on one day at 20 mg/kg for four times at 2-hour intervals, as previously described (4). Beginning one week later, mice received a saline or TZ injection once a day. At the end of the drug or saline treatment, behavioral tests including rotarod test and pole test were carried out before the animals were sacrificed. As for the other drug tests (urapidil, tamsulosin, doxazosin, alfuzosin and prazosin), the experimental design was the same as for TZ.

Unilateral 6-OHDA Lesion in Rats.

For the 6-OHDA model in rats, pentobarbital sodium (80 mg/kg) was used as anesthesia by i.p. injection. Then, the rats were fixed in a stereotaxic frame (Benchmark, myNeuroLab, S-072607). 6-OHDA was dissolved in 0.2% ascorbic acid saline solution at 2.5 μg/μ1. Unilateral injection of 6-OHDA was performed according to the stereotaxic atlas of rat (5). 6-OHDA was injected into two sites in the right striatum, with 10 μg for each site (coordinates with respects to bregma: AP, +0.8 mm; ML, +2.7 mm; DV, −5.2 mm; and AP, +0.8 mm; ML, +2.7 mm; DV, −4.5 mm) at a rate of 1 μl/min. using a 10-μl Hamilton syringe (6). The same amount of saline was injected the same way as a control. After the injection, the needle was left at the last site for another 5 min. before slow retraction. After the surgery, rats were placed on a warm electric blanket for recovery. Two weeks, three weeks, four weeks or five weeks later, apomorphine-induced rotational behavior was assessed to select the rats that had been successfully targeted. They were then randomly divided into two groups: saline treatment group and TZ treatment group (70 μg/kg i.p.). Based on the most effective doses of TZ at 10 and 100 μg/kg in mice, we selected TZ at 70 μg/kg in rats. Sham-operated animals received saline in the same way. All these three groups were treated with saline or TZ for two weeks followed by locomotor activity assessment. After behavior testing, animals were sacrificed.

Two weeks after stereotaxic surgery, 6-OHDA-treated rats were given 0.5 mg/kg apomorphine (i.p.) (7). Then, the rats were placed in a transparent cylinder (diameter 30 cm, height 35 cm). Five min. later, contralateral rotation behavior was measured for 30 min. and recorded with a camera. The rats with a rotation rate over 7 turns/min. were selected for further studies (8).

Rotarod Test.

The rotarod test was carried out using an automated rotarod (E103, UGO BASILE). At a fixed speed of 15 revolutions per minute (rpm), mice were pre-trained for two consecutive days until they were able to remain on the rod for more than 60 seconds. On the 7^{th} day and the 14^{th} day after MPTP injection, mice were tested on the rotating rod at an acceleration mode (2-45 rpm). The latency to fall was recorded for a maximum recording time of 600 seconds. The behavior was monitored by a video camera.

Pole Test in Mice.

This test was carried out by leaving a pole in the cage where the mice were housed. The pole test was performed on the 14^{th} day after MPTP injection as previously described (9). The mouse was placed in a head-upward position on top of a vertical pole (diameter 8 mm, height 55 cm) with a ball (diameter 2.5 cm) on the head of the pole. The pole was wrapped with the nylon gauze to prevent the mouse from slipping down. Each mouse was trained twice before testing. The time that the mouse took to turn his head from upward to downward (Time: Turn) and the time the mouse took to reach the floor with his forepaws (Time: Locomotion Activity) were recorded. Each mouse was tested three times with 5 min. intervals, and the average time was quantified.

Cylinder Test in Rats.

Forelimb movement coordination of rats was analyzed by the cylinder test as previously described (10). The rats were individually placed in a transparent cylinder (diameter 30 cm, height 35 cm). After 5 min. adaptation, their wall-contact with left, right or both fore-paws was counted until the total number of wall-contacts was 20. Then the percentage of left, right or both fore-paws touching were analyzed.

Immunohistochemistry and Immunofluorescence.

After anesthesia with pentobarbital sodium (80 mg/kg), animals were perfused with 0.9% saline followed by 4% ice-cold paraformaldehyde (PFA) in 0.1 M phosphate buffer (PBS, pH 7.4) as previously described (11). Brains were removed, post-fixed in PFA overnight, and transferred into 10%, 20% and 30% sucrose until brains successively sank to the bottom. Brains were cut into 30 μm thick coronal slices (in six series), free-floating sections were rinsed in PBS for three times, and quenched in 3% H₂O₂ for 10 min. Sections were pre-incubated in 2% BSA/0.3% triton x-100 in PBS (0.3% PBST) for one hour at room temperature, followed by incubation with primary antibody in the blocking solution overnight. To detect DA neuron cell bodies in the SNc and their fibers in the striatum, the rabbit anti-tyrosine hydroxylase (1:2000, AB152, Millipore) antibody was used. After three 10 min. washes with 0.3% PBST, brain sections were incubated with corresponding biotinylated secondary antibody (1:200, BE0101, EasyBio Technology Co., Ltd.), and subsequently incubated with avidin-biotin-peroxidase complex for one hour at room temperature. Then, the brain sections were treated with 3, 3′-diaminobenzidine and 0.01% H₂O₂ for 1-5 min. After dehydration in gradient alcohol and clearing in xylene, the brain slices were mounted on lysine pre-treated glass slides and cover-slipped in DPX (DPX mountant for histology). The brain slices were imaged under a stereoscope, and TH neurons and their fibers were analyzed using Stereo Investigator software (version 8) and Image-pro Plus 6.0, respectively. For immunofluorescence of the animal brain slices, the experimental procedures were similar to the immunohistochemistry protocol.

For immunofluorescence of iPSC-derived cells, cells were fixed with 4% paraformaldehyde (PFA) in PBS for 20 min. and blocked in 0.3% Triton X-100 (Sigma-Aldrich, Madrid, Spain) with 3% donkey serum for 2 hr, followed by incubation with primary antibodies 4° C. overnight and secondary antibodies at room temperature for 2 hr. In the case of α-synuclein staining, Triton X-100 was kept at 0.01% for the blocking and antibody incubation steps. Images were acquired using a Leica SP5 confocal microscope. We also performed a Sholl analysis, a widely used method to quantify the complexity of dendritic arbours. A Sholl profile is obtained by plotting the number of dendrite intersections against the radial distance from the soma center (12).

Western Blot Analysis.

For protein detection, corresponding brain regions were harvested immediately after animals were euthanized and stored at −80° C. before protein extraction. RIPA buffer (Beyotime, Beijing, China) containing a cocktail of protease inhibitors (Roche, Mannheim, Germany) and PMSF (Sigma) were used for protein extraction according to a standard protocol (13). After disruption on ice for 30 min., the lysates were ultrasonicated and then centrifuged at 12,000 rpm for 15 min., and supernatants were harvested. Samples were separated on 12% SDS-polyacrylamide gels followed by transfer to PVDF membranes (Millipore, Mass., USA). Membranes were blocked with 5% nonfat milk for one hour at room temperature and incubated with the primary antibody overnight at 4° C. Membranes were washed 3 times for 10 min. with 0.1% Tween-20/PBS and then incubated with an IRDye 700 or 800-labeled secondary antibody (1:10000), and scanned with an Odyssey infrared imaging system (LI-COR instrument, Lincoln, Nebr., USA). The target protein levels were normalized to β-actin levels. The results were analyzed using the ImageJ2X software.

Striatal DA Content Detection.

A high-performance liquid chromatography with electrochemical detector (HPLC-ECD) was used to detect the dopamine content as previously reported (14). Each animal tissue was accurately weighed and homogenized in 200 μl ice-chilled 0.1 M perchloric acid. The homogenate was centrifuged at 12,000 rpm for 20 min. at 4° C., 160 μl of supernatant was collected and mixed with 80 μl ice-chilled solution B (20 mM potassium citrate, 300 mM dipotassium hydrogen phosphate, and 2 mM disodium ethylenediaminetetraacetate (EDTA-2Na), then centrifuged again and the supernatant was injected in HPLC for determination of catecholamines. Dopamine, DOPAC, and HVA content were reported as μg per mg wet tissue and normalized to the control group.

Assay of TZ in Rat Blood and CSF.

HPLC-ECD was used to detect the TZ content in rat blood and cerebral spinal fluid (CSF). Rats were given 30 mg/kg TZ (i.p.), and blood and CSF samples were collected 20 min. after drug administration following a previous protocol (15). CSF was immediately preserved at −80° C., while blood was equilibrated for 20 min. at room temperature and then centrifuged again for 15 min. at 4500 rpm, the supernatant was collected and stored at −80° C. Blood and CSF samples were filtered using a 0.22 μm filter and 50 μl was used for detection. Standard curves were prepared with known amounts of TZ in double distilled water, yielding concentrations of 0, 1, 10 and 50 μg/ml. The content of TZ in CSF was divided by the TZ content in blood.

Mitochondrial DNA Content.

Relative mitochondria content can be estimated by the 16s rRNA and ND1 (NADH dehydrogenase 1, a mitochondrial protein) (16). Mitochondrial DNA (mtDNA) was extracted from mouse brain tissues. After a rinse in PBS, tissues were placed in an ice-cold 1.5-ml microcentrifuge tube. 600 μl of lysis buffer (RIPA) was added to the tube, followed by 0.2 mg/ml proteinase K, to degrade the proteins present in the tissue sample. Then, samples were incubated at 55° C. for 3 hr. 100 μg/ml RNase A was added to degrade the RNA, incubated at 37° C. for 30 min. 250 μl of 7.5 M ammonium acetate and 600 μl of isopropanol were added, and mixed well. Samples were centrifuged for 10 min. at 15,000×g at 4° C., and the supernatant was removed. Pellets were washed with 500 μl 70% ethanol and dried for 10 min. Then the pellets were resuspended in 100 μl of TE buffer. The concentration of mtDNA was measured using a NanoDrop spectrophotometer, and a final concentration of 10 ng DNA/μl was used for qPCR.

Drosophila Stock and Rotenone Toxicity Assay.

The flies used in this study included w¹¹¹⁸, PINK1⁵, LRRK^{ex1}, TH-Gal4, Appl-Gal4, Mhc-Gal4 and Actin-Gal4 purchased from Bloomington Fly Stock Center and Pgk RNAi (Tsinghua TRiP RNAi stock, THU0568) purchased from Tsinghua TRiP RNAi stock. The UAS-Pgk transgene was generated by P-element insertion under the w¹¹¹⁸ background by our laboratory. For all experiments, the flies were maintained in an incubator set with 25° C. and 60% humidity under a 12 hr light/dark cycle.

For the rotenone assay, 20 flies at 1-3 days old were collected and placed in each vial; for each experimental condition, 10 vials were tested. Rotenone (125 μM and 250 μM) were mixed in cornmeal fly food. The vial was replaced with a new vial every two days for a week. To assess behavioral performance, the flies were transferred into a transparent tube (height, 40 cm; diameter, 1.5 cm). Then, these flies were gently tapped to the bottom of the tube. Flies climbing past the 25 cm mark in 20 sec. were recorded as normal motor behavior (17).

PINK1⁵ male flies were treated with TZ at 0 μM, 0.1 μM, 1 μM and 10 μM for 10 days from the 1^{st} day after eclosion or TZ at 1 μM for 7 days from the 3^{rd} day after eclosion. Wing defects were recorded every day or just at the end of TZ treatment depending on the experimental design. For TH neurons immunostaining and western blots, TZ was given to the adult flies after eclosion. After 18-20 days, fly heads were harvested. The PPL1 cluster of TH neurons were immunostained and counted.

LRRK^{ex1} male flies were treated with TZ (1 μM) for 10 days after eclosion. The wing defects were recorded after 10 days of treatment.

Glycolysis and Mitochondrial Stress Measured by XF-24 Seahorse Assays.

A Seahorse XV analyzer (XF^{e}-24, Seahorse Bioscience, Billerica, Mass., USA) was used to measure the oxygen consumption rate (OCR) and the extracellular acidification rate (ECAR) as previously described (18). The M17 cells and iPS cells were seeded in XF^{e} 24-well plates (Seahorse Bioscience), while the plates used for iPS cells were poly-D-lysine pre-coated. Assay medium was prepared by supplementing Seahorse XF^{e}BaseMedium minimal DMEM (Seahorse Bioscience) with 2 mM L-glutamine for a Glycolysis Stress Test assay or 2 mM L-glutamine, 1 mM pyruvate and 10 mM glucose for a Mito Stress Test assay (Sigma). pH was adjusted to 7.4 at 37° C. Probes (Seahorse Bioscience) were hydrated in the calibrant (Seahorse Bioscience) in a non-CO₂ incubator at 37° C. overnight. Cells were washed twice with assay medium and kept in a non-CO₂ incubator at 37° C. for 1 hr before analysis. Glucose, oligomycin and 2-deoxy-glucose (2-DG) were pre-loaded in the probe plate for Glycolysis Stress Test, while oligomycin, FCCP and a mixture of rotenone and antimycin A were used for Mito Stress Test.

ATP Assay, Citrate Synthase Activity, LDH Assay and Pyruvate Level Detection.

Citrate synthase (CS) activity, LDH assay and pyruvate level were detected using commercial kits according to the manufacturer's directions. ATP content in animal tissues and M17 cells were detected with the ATP assay kit following the manufacturer's directions. ATP production by iPSCs was measured with the ATP Determination Kit (A22066, Molecular Probes) following the manufacturer's directions. 24 hr after plating, iPSC-derived neural progenitors were treated with 10 μM TZ for 24 hr. Cells were then washed with dPBS and detached with EDTA (AM9260G, Thermo Scientific) for counting. After washing them with ice-cold PBS, cells were centrifuged, and the supernatant was discarded. ATP buffer (100 nM Tris-HCL pH 7.75, 4 mM EDTA) was added. Cells were then flash frozen in liquid nitrogen, followed by a 3-min. boil, and 5 min. on ice. Cells were centrifuged at 4° C. for 5 min. at 13,000 rpm. The supernatant was used with the ATP determination kit. Each reaction contained 1.25 μg/ml of firefly luciferase, 50 μM D-luciferin and 1 mM DTT in 1× Reaction Buffer. After 15 min. incubation, luminescence was measured and the production of ATP per cell calculated.

Nissl Staining.

The brain slices of the striatum region were harvested for Nissl staining according to the protocol described above. Coronal slices (in six series) were mounted on lysine pre-treated glass slides, and dehydrated in gradient alcohol, cleared in xylene, cover-slipped in DPX followed by Nissl staining for 30 min. at room temperature. For neuron counting, six fields were randomly selected in one slice and six slices were used for each brain, three animals were counted for each group.

Tunel Assay.

Mice and rat brain coronal slices (in six series) were collected for TUNEL assay, which was performed according to the manufacturer's protocol (Promega). Brain slices were fixed in 4% PFA for 15 min. at 15-25° C., washed 3 times with PBS. Sections were incubated in permeabilization solution (0.3% triton x-100 in PBS) for 15 min. at 15-25° C. Then, slices were treated with proteinase K (10 μg/ml) for 10 min. at 56° C., followed by fixed in 4% PFA for 15 min. at 15-25° C., and rinsed in PBS three times. TUNEL reaction buffer was added and incubated for 2 hr at 37° C. in a humidified atmosphere in the dark. After rinsing in PBS three times, samples were analyzed in a drop of PBS under a fluorescence microscope using an excitation wavelength in the range of 450-500 nm and detection in the range of 515-565 nm.

TUNEL/TH Co-Staining Assay.

Following immunohistochemistry, TUNEL was detected with the In Situ Cell Death Detection Kit (Roche Diagnostics, USA). Mouse brain slices were incubated with TUNEL reaction buffer for 2 hr at 37° C. After rinsing with PBS 3 times, the samples were analyzed under a confocal microscope (Leica SP8, Germany).

RNA Extraction and qRT-PCR.

Flies of actin>Pgk RNAi and actin>attp2 (as a control) were harvested, and total RNA was extracted using Trizol reagent according to the manufacturer's instructions (Invitrogen Life Technologies, CA, USA). Two μg RNA were reverse transcribed using the RevertAid First Strand cDNA Synthesis kit according to the manufacturer's protocol (Thermo Scientific). Real-time PCR analysis was performed followed the standard protocol from Applied Biosystems (7500 real-time PCR system, ABI Inc.). Actin was used as a reference for total RNA quantity.

Cell Culture Experiments with iPSC Cell Lines Derived from Human Patients.

All procedures adhered to Spanish and EU guidelines and regulations for research involving the use of human pluripotent cell lines. The human iPSC lines used in our studies were generated following procedures approved by the Commission on Guarantees concerning the Donation and Use of Human Tissues and Cells of the Carlos III Health Institute, Madrid, Spain.

The human iPSC lines SP11 (from control), and SP12 and SP13 (from patients with familial PD carrying the LRRK2^{G2019S} mutation) have been previously described (19). iPSC culture and differentiation toward midbrain DA neurons was carried out as described (20), following procedures approved by the Spanish competent authorities (Commission on Guarantees concerning the Donation and Use of Human Tissues and Cells of the Carlos III Health Institute). Briefly, iPSC were cultured on Matrigel (Corning Limited, Life Sciences, UK) and maintained in hESC medium, consisting of KO-DMEM supplemented with 20% KO-Serum Replacement, 2 mM Glutamax, 50 μM 2-mercaptoethanol (all from Invitrogen, Thermo Fisher Scientific, Madrid, Spain), non-essential amino acids (Cambrex, Nottingham, UK), and 10 ng/ml bFGF (Peprotech, London, UK). Medium was preconditioned overnight by irradiated mouse embryonic fibroblast and iPSC were cultured at 37° C., 5% CO₂. For midbrain DA neuron differentiation, iPSC were disaggregated with Accutase and embryoid bodies (EB) generated using forced aggregation in V-shaped 96-well plates. Two days later, EBs were patterned as ventral midbrain by culturing them in suspension for 10 days in N2B27 supplemented with 100 ng/ml SHH, 100 ng/ml FGF8, and 10 ng/ml FGF2 (all from Peprotech, London, UK). Then, for α-synuclein and neurite analysis, differentiation to midbrain DA neurons was performed on the top of PA6 murine stromal cells for 3 weeks (PMID: 21877920). TH positive neurons in normal control was ˜70%, and ˜55% in subject 12 and 45% in subject 13 with LRRK^{G2019S} mutations. To analyze α-synuclein levels, neuronal cultures were gently trypsinized and re-plated on Matrigel-coated slides. One day and three days after plating, DA neurons were treated for 24 hr with 10 μM TZ, after which cells were fixed and analyzed.

Immunofluorescence Analysis of iPSC-Derived Cells.

iPSC-derived cells were fixed with 4% paraformaldehyde (PFA) in Tris-buffered saline (TB S) for 20 min. and blocked in 0.3% Triton X-100 (Sigma-Aldrich, Madrid, Spain) with 3% donkey serum for 2 hr. In the case of α-synuclein staining, Triton X-100 was kept at 0.01% for the blocking and antibody incubation steps. The following primary antibodies were used: mouse anti-α-synuclein (610787, BD Biosciences, Madrid, Spain), rabbit anti-TH (sc-14007, Santa Cruz Biotechnology, Madrid), and mouse anti-TUJ1 (801202, Biolegend). Images were acquired using a Leica SP5 confocal microscope.

Analysis of the Parkinson's Progression Markers Initiative (PPMI) Database.

We analyzed data from the PPMI database (21) for patients taking TZ alone, TZ/DZ/AZ, or tamsulosin. We tested if these drugs influenced the rate of motor progression as measured by part III of the Movement Disorder Society's Unified Parkinson's Disease Rating Scale (MDSUPDRS), which is a metric of motor disability in Parkinson's disease (22). For this analysis, only participants who were using TZ/DZ/AZ or tamsulosin at their baseline PPMI visit were included in the drug groups. PPMI protocol dictates that the fourth visit should occur approximately one year after the baseline visit; accordingly, any visit that occurred between the baseline visit and the fourth PPMI visit were included. Participants also had to have more than one visit to be included. Of the 13 participants in the TZ/DZ/AZ group, 11 were taking the medication-of-interest without breaks until their fourth visit. One participant was taking DZ at the time of their baseline visit, but discontinued within 30 days of their baseline visit. That participant was only considered to be using DZ during their first and second visits. Another participant was using AZ at their baseline visit and for approximately 5 months after that. This participant was considered to be taking AZ during their first and second visits. If these two participants were excluded from the analysis and only participants who were taking the medication-of-interest without breaks in therapy were included, the results change only marginally. The TZ/DZ/AZ group (n=11) has a slope of change of 0.02±0.21 compared to 0.53±0.05 in the control group (n=269, p=0.015). Only male participants were included as all patients taking TZ/DZ/AZ and tamsulosin were males. The indication for TZ/DZ/AZ and tamsulosin in all patients was benign prostatic hyperplasia or undefined urological problems. The characteristics of the patients are shown in Table 1.

Only MDS-UPDRS readings that were obtained when the participants were not yet taking a PD medication or were in the practically defined OFF state (at least 6 hours after the last dose of levodopa or any other anti-PD medication) were utilized for this analysis. We employed linear mixed effect regression (LMER) analyses to evaluate any differences in the slopes of the relative UPDRS scores between patients who were taking TZ, TZ/DZ/AZ, or tamsulosin compared to those who were not taking TZ/DZ/AZ. An unadjusted model was initially constructed that included the MDS-UPDRS Part 3 score as the dependent variable and the duration * medication group interaction term as the independent variable. The model also allowed random intercepts per subject as well as differing slopes of time for each subject. In this unadjusted model, the monthly increase in the MDS-UPDRS Part 3 score in the control group was 0.31±0.04 compared to a monthly decrease in MDS-UPDRS Part 3 of −0.21±0.21 in the TZ/DZ/AZ group (p=0.013). We then constructed a similar model that was aimed to include covariates that may predict progression of the MDS-UPDRS Part 3 score over time. This model included age at baseline, age of symptom onset, use of PD medications at each visit, baseline MDS-UPDRS Part 3 score, and baseline Hoehn & Yahr score. In this adjusted model, the monthly increase in the MDS-UPDRS Part 3 score in the control group was 0.54±0.05 compared to a monthly increase in MDS-UPDRS Part 3 of 0.04±0.22 in the TZ/DZ/AZ group (p=0.022). Maximum likelihood methods were used to test differences in the intercepts and the slopes between groups. R was utilized for all analyses.

Analysis of the IBM Watson/Truven Database

Cohort Identification.

We identified male enrollees in the Truven Health Marketscan Commercial Claims and Encounters and Medicare Supplemental databases that had at least one outpatient diagnosis of Parkinson's disease (ICD-9 332.0, ICD-10 G20) between 2011 and 2016 and who were prescribed terazosin, doxazosin, or alfuzosin (TZ/DZ/AZ) or tamsulosin (control). Analysis was restricted to the initial period of uninterrupted time when an enrollee was plausibly taking one of the 4 drugs. Table 2 shows the numbers of enrollees, the person years of drug exposure, the age, and the average drug dosage.

ICD-9 to ICD-10 Translation.

The ICD-9 to ICD-10 changeover happened on 2015 Oct. 1. Approximately 25% of the diagnoses codes in our data are from ICD-10 while the rest are ICD-9 codes. Due to the relatively recent introduction of ICD-10, little work has previously been done using ICD-10 codes relative to the ICD-9 standard. To that end, we started by using only ICD-9 codes and then used the Centers for Medicare and Medicaid Services (CMS) ICD-9 to ICD-10 crosswalk as provided by the National Bureau of Economic Research. Recent publications have shown that the translations provided by this file are generally complete and reasonable translations, at least in the domain of cardiovascular outcomes (23).

Identifying Codes in PD Patients and PD-Related Codes.

We identified the top 500 most commonly occurring ICD-9 codes among the cohort, regardless of whether or not enrollees were taking one of the drugs of interest (TZ/DZ/AZ and tamsulosin at the time). This comprised a set of common diagnostic codes that we used to search for differences in relative incidence. Days on which enrollees had the relevant diagnosis code were identified by matching the ICD-9 diagnosis code directly (2011 Jan. 1 to 2015 Sep. 31) or matching the crosswalked ICD-10 diagnosis code (2015 Oct. 1 to 2016 Dec. 31). Of the 500 considered codes, 497 occurred at least 50 times in the tamsulosin group and at least 50 times in the TZ/DZ/AZ group during the study period and were therefore included in the model.

The most frequent 497 ICD-9 codes were also reviewed by two neurologists whose clinical practice focuses on PD. Without knowledge of results, they labeled each code as either potentially PD-related or unrelated to PD. A total of 80 codes were identified as PD-related. Because code 332.0 (Paralysis agitans) was used to identify patients with PD, we excluded that code from further analysis. That left us with a group of 79 PD-related codes. Those codes were further grouped as being motor, non-motor, or complications.

Defining Medication Days.

We were interested in defining days when the enrollee had the medication and was plausibly taking the medication. We started by considering the proportion of days covered (PDC) measure of adherence. The PDC is simply the ratio of the number of days supplied provided in a dispensing event and the number of days until the next dispensing event for that medication (24, 25). A threshold of 80% is commonly considered “adherent to therapy” for medications used to manage diabetes and cardiovascular disease and is the threshold we selected here (26). A PDC of 80% corresponds to a refill occurring no more days later than 125% of the days supplied. For example, if a filled prescription (fill) had a 30 day supply, in order to have a PDC of at least 80% we would require a refill within 37.5 days (30/37.5=0.80). We identified each dispensing event and coded the following 125% of days supplied days as “taking the medication.” After constructing this exposure variable for each fill, we constructed a variable for each person-day that took the value 1 if it was within 125% days supplied of any fill and 0 otherwise.

For each enrollee, we only used data from the first observed medication period. We chose not to include data from periods after the medication was potentially stopped and later restarted because the reason for changes in medication would be unknown and potentially could introduce confounding. We defined the first medication period to be all fills after the first fill such that there was no interval between fills longer than (125% of days supplied)+90 days. We discarded any data after the first interval longer than this threshold between fills.

Analysis of the Codes.

The effect of TZ/DZ/AZ vs. tamsulosin was estimated using a generalized linear model (GLM) with a quasi-Poisson distribution. The model is given by:

$${n}_{\text{?}} = {β}_{0} + {β}_{1} {d}_{i} + log ( {t}_{\text{?}} ) + {ϵ}_{i}$$

$$\text{?} \text{indicates text missing or illegible when filed}$$

where nᵢ is the number of days on which the i^{th} enrollee had an outpatient visit with the diagnosis code of interest, dᵢ takes the value of 1 if the enrollee was taking TZ/DZ/AZ or terazosin and 0 if the enrollee was taking tamsulosin, tᵢ is the total number of days the enrollee was taking that medication class, and ∈ᵢ is a mean zero error term. The value of log(tᵢ) is included as an offset to account for different durations of observation between enrollees and is logged to match the link function expected by the quasi-Poisson distribution.

We elected to use a quasi-Poisson GLM over a classic Poisson GLM to allow for over-dispersion of the data. A Poisson distribution assumes that the mean and variance are equal. In practice, it is quite common for the variance of a count variable to be very different from the mean. The quasi-Poisson GLM extends the classic Poisson GLM by assuming that the variance can be written as the product of a scalar multiplier and the mean. This allows the data to have a larger variance than would be permitted under the classic Poisson framework.

We estimated relative risks for the 497 tested codes, 166 (33.4%) had a significantly different incidence between the groups at p=0.05. Of those, 43 (25.9%) were plausibly PD-related.

In the code-by-code model described above, we considered the incidence of each code separately and independently; however, there are clinically meaningful clusters of codes where pooling may increase the analytic power. The two neurologists labeled each PD related code as being a motor symptom, a non-motor symptom or a complication of PD and within those three large groups we further clustered codes into clinically meaningful groups or by organ system. We counted the number of days for each person where they had at least one of the codes in the sub-group and modeled this count using the same quasi-Poisson model described above.

Incidence and Survival Analysis.

A cohort of enrollees newly started on TZ/DZ/AZ or tamsulosin was constructed. We defined newly started as at least 365 days of enrollment with prescription drug coverage prior to the first fill event for TZ/DZ/AZ or tamsulosin. Additionally, we required the enrollee to be PD-free at the time of the first fill (no prior PD diagnosis code).

A total of 78,509 enrollees on TZ/DZ/AZ were identified and these enrollees were followed for an average of 285±382 days with a total of 118 cases of PD (incidence=0.15%). We matched each TZ/DZ/AZ user to a tamsulosin user of the same age at medication start and with the minimum difference in the duration of follow up. We were able to successfully match 78,444 of the 78,509 enrollees on TZ/DZ/AZ to an enrollee on tamsulosin. In the matched cohort, enrollees taking TZ/DZ/AZ have, on average, 284±381 days of follow up compared to 284±382 days of follow up in those taking tamsulosin. Of the 78,444 enrollees taking TZ/DZ/AZ, a total of 118 (0.15%) developed PD compared to 190 (0.24%) among those taking tamsulosin.

We used a Cox proportional hazards regression to model the relative hazard of developing PD among those in the matched cohort taking TZ/DZ/AZ compared to those taking tamsulosin while accounting for censoring due to stopping the medication or exiting the data before developing PD. This model estimated a hazard ratio for those taking Az/Dz/Tz to be 0.62 (95% CI: 0.49-0.78).

Data Availability.

Data and code for PPMI and Truven data are available at narayanan.lab.uiowa.edu/datasets.

Statistical and Analysis Considerations.

For experiments to quantify animal behavior and for sample collections, experimenters were blinded to genotype and intervention, and studies were done by two different experimenters. Numbers of animals studied were based on our past experience and preliminary data. In all figures, data points are from individual mice and rats, or groups of flies. We did not exclude any data points from this study. Bars and whiskers indicate mean±SEM. Blue indicates controls and red indicates TZ treatment. Statistical significance for comparisons between data sets was primarily with non-parametric tests. For studies of fly motor performance, our previous studies showed that within a group of flies (15-50 flies for one data point), the data fit a gaussian distribution. Moreover, multiple groups of flies also fit a gaussian distribution. Therefore, parametric tests were used to evaluate statistical significance. Table S5 shows the statistical test used for all data and the resulting P value for comparisons. All statistical tests were two-tailed. On individual graphs, we show statistical significance for the main comparisons with asterisks *p<0.05, **p<0.01, ***p<0.001.

### SUPPLEMENTAL REFERENCES

1. Liu Z Y, Hu S P, Ji Q R, Yang H B, Zhou D H, and Wu F F. Sevoflurane pretreatment inhibits the myocardial apoptosis caused by hypoxia reoxygenation through AMPK pathway: An experimental study. Asian Pac J Trop Med. 2017; 10(2):148-51.
2. Chen H C, Lee J T, Shih C P, Chao T T, Sytwu H K, Li S L, et al. Hypoxia Induces a Metabolic Shift and Enhances the Stemness and Expansion of Cochlear Spiral Ganglion Stem/Progenitor Cells. Biomed Res Int. 2015; 2015:359537.
3. Rockenstein E, Mallory M, Hashimoto M, Song D, Shults C W, Lang I, et al. Differential neuropathological alterations in transgenic mice expressing alpha-synuclein from the platelet-derived growth factor and Thy-1 promoters. J Neurosci Res. 2002; 68(5):568-78.
4. Sonsalla P K, and Heikkila R E. The influence of dose and dosing interval on MPTP-induced dopaminergic neurotoxicity in mice. Eur J Pharmacol. 1986; 129(3):339-45.
5. Paxinos G, and Watson C. The rat brain in stereotaxic coordinates. Sydney Academic Press. 1982.
6. Kirk D, Rosenblad C, and Bjorklund A. Characterization of behavioral and neurodegenerative changes following partial lesions of the nigrostriatal dopamine system induced by intrastriatal 6-hydroxydopamine in the rat. Exp Neurol. 1998; 152(2):259-77.
7. Baluchnejadmojarad T, Roghani M, and Mafakheri M. Neuroprotective effect of silymarin in 6-hydroxydopamine hemi-parkinsonian rat: involvement of estrogen receptors and oxidative stress. Neurosci Lett. 2010; 480(3):206-10.
8. Song L, Zhang Z, Hu R, Cheng J, Li L, Fan Q, et al. Targeting the D1-N-methyl-D-aspartate receptor complex reduces L-dopa-induced dyskinesia in 6-hydroxydopamine-lesioned Parkinson's rats. Drug Des Devel Ther. 2016; 10:547-55.
9. Luchtman D W, Shao D, and Song C. Behavior, neurotransmitters and inflammation in three regimens of the MPTP mouse model of Parkinson's disease. Physiol Behav. 2009; 98(1-2):130-8.
10. Laganiere J, Kells A P, Lai J T, Guschin D, Paschon D E, Meng X, et al. An engineered zinc finger protein activator of the endogenous glial cell line-derived neurotrophic factor gene provides functional neuroprotection in a rat model of Parkinson's disease. J Neurosci. 2010; 30(49): 16469-74.
11. Zhang F L, He Y, Zheng Y, Zhang W J, Wang Q, Jia Y J, et al. Therapeutic effects of fucoidan in 6-hydroxydopamine-lesioned rat model of Parkinson's disease: Role of NADPH oxidase-1. CNS Neurosci Ther. 2014; 20(12):1036-44.
12. Binley K E, Ng W S, Tribble J R, Song B, and Morgan J E. Sholl analysis: a quantitative comparison of semi-automated methods. J Neurosci Methods. 2014; 225:65-70.
13. Tsui P, Telischi M, and Tsang V C. A rapid and economical method for the identification of anti-HIV antibodies by the western blot technique. Biotechniques. 1988; 6(5):400-2.
14. Xu G, Xiong Z, Yong Y, Wang Z, Ke Z, Xia Z, et al. Catalpol attenuates MPTP induced neuronal degeneration of nigral-striatal dopaminergic pathway in mice through elevating glial cell derived neurotrophic factor in striatum. Neuroscience. 2010; 167(1):174-84.
15. Liu L, and Duff K. A technique for serial collection of cerebrospinal fluid from the cisterna magna in mouse. J Vis Exp. 2008(21):pii: 960.
16. Quiros P M, Goyal A, Jha P, and Auwerx J. Analysis of mtDNA/nDNA Ratio in Mice. Curr Proctoc Mouse Biol. 2017; 7(1):47-54.
17. Friggi-Grelin F, Coulom H, Meller M, Gomez D, Hirsh J, and Birman S. Targeted gene expression in Drosophila dopaminergic cells using regulatory sequences from tyrosine hydroxylase. J Neurobiol. 2003; 54(4):618-27.
18. Lee H T, Lin C S, Pan S C, Wu T H, Lees C S, Chang D M, et al. Alterations of oxygen consumption and extracellular acidification rates by glutamine in PBMCs of SLE patients. Mitochondrion. 2018; pii: 51567-7249(17):30195-2.
19. Sánchez-Danés A, Richaud-Patin Y, Carballo-Carbajal I, Jimenez-Delgado S, Caig C, Mora S, et al. Disease-specific phenotypes in dopamine neurons from human iPS-based models of genetic and sporadic Parkinson's disease. EMBO Mol Med. 2012; 4(5):380-95.
20. Sánchez-Danés A, Consiglio A, Richaud Y, Rodríguez-Pizà I, Dehay B, Edel M, et al. Efficient generation of A9 midbrain dopaminergic neurons by lentiviral delivery of LMX1A in human embryonic stem cells and induced pluripotent stem cells. Hum Gene Ther. 2012; 23(1):56-69.
21. Marek K, Jennings D, Lasch S, Siderowf A, Tanner C, Simuni T, et al. The Parkinson Progression Marker Initiative (PPMI). Prog Neurobiol. 2011; 95(4):629-35.
22. Disease MDSTFoRSfPs. The Unified Parkinson's Disease Rating Scale (UPDRS): status and recommendations. Mov Disord. 2003; 18(7):738-50.
23. Columbo J A, Kang R, Trooboff S W, Jahn K S, Martinez C J, Moore K O, et al. Validating Publicly Available Crosswalks for Translating ICD-9 to ICD-10 Diagnosis Codes for Cardiovascular Outcomes Research. Circ Cardiovasc Qual Outcomes. 2018; 11(10):e004782.
24. Raebel M A, Schmittdiel J, Karter A J, Konieczny J L, and Steiner J F. Standardizing terminology and definitions of medication adherence and persistence in research employing electronic databases. Med Care. 2013; 51(8 Suppl 3):S11-21.
25. https://www.ncpanet.org/pdf/adherence_list.pdf.
26. https://www.pqaalliance.org/adherence-measures.

### Example 2—Additional Data Indicate that TZ, AZ, and Related Chemicals can Benefit Other Neurodegenerative Diseases in Addition to Parkinson's Disease (PD

The FUS Protein Associated with Amyotrophic Lateral Sclerosis (ALS).

We studied Hela cell stably expressed FUS-GFP to determine whether alfazosin (AZ) affected the molecule biology of FUS. (See FIG. 19). FUS forms phase-separated aggregates in cells and is commonly used to test agents that enhance or inhibit aggregate formation. The cells were maintained under mild hypoxic condition (5% oxygen), in which mitochondria function at their highest level. Alfazosin (AZ) was added in the culture medium with or without rotenone, a mitochondrial respiration inhibitor. 60 hours later, we measured the ATP level. The control (without AZ) is set as 1 (N=3). The data show that AZ increases ATP levels, and this effect depends on mitochondrial function, because blocking mitochondrial respiration abolished the ATP increase.

AZ also reduced FUS-GFP protein levels, and this effect depends on both mitochondrial function and HSP90. We quantified FUS-GFP protein levels using actin as a protein loading control. (See FIG. 20). Rotenone (Rot) is a mitochondrial respiration inhibitor, 17AAG is an inhibitor of the ATPase of HSP90. The control level of FUS/actin is set as 1, and relative ratio of other treatment is compared.

Fluorescence recovery after photobleaching shows that FUS-GFP has greater mobility in cells treated with AZ. (See FIG. 21). After 30 seconds photobleaching, the recovery of GFP signal was measured and plotted. N=10. These results suggest that AZ reduces the phase separation of FUS-GFP protein in cells.

The Amyloid Precursor Protein (APP) Associated with Alzheimer's Disease.

We also studied Hek293T cells that were transfected with the amyloid precursor protein (APP) Swedish mutation tagged with GFP (APPswe-GFP). (See FIG. 22). After transfection, AZ (1-10 μM) was added for 24 hours. The GFP intensity without AZ treatment was set as 1. AZ was observed to decrease the aggregation of APPswe-GFP.

AZ (1-100 μM) was added for 24 hours to Hek293T cells expressing APPswe-GFP. Then, cell lysates were collected for protein quantification by western blot. (See FIG. 23). The APP/actin ratio was plotted. This results suggest that AZ reduces the APPswe-GFP levels in cells.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A method for treating and/or preventing a neurodegenerative disease or disorder or symptoms thereof in a subject in need thereof selected from the group consisting of Alzheimer's disease (AD), Huntington's disease (HD), amyotrophic lateral sclerosis (ALS), Lewy body dementia, the method comprising administering to the subject a pharmaceutical composition comprising an effective amount of a therapeutic agent that activates phosphoglycerate kinase 1 (PGK1) selected from the group consisting of terasozin, prazosin, doxozosin, alfuzosin, trimazosin, and abanoquil or pharmaceutical salts or hydrates thereof.

2. The method of claim 1, wherein the therapeutic agent is formulated as a time-release preparation.

3. A method for treating and/or preventing a neurodegenerative disease or disorder in a subject in need thereof, the method comprising administering to the subject a pharmaceutical composition comprising an effective amount of a therapeutic agent that activates phosphoglycerate kinase 1 (PGK1) and preferably does not bind to the α₁-adrenergic receptor (α₁AR) or preferably binds to the α₁AR but with a dissociation constant (K_{d}(α₁AR)) greater than about 10 μM.

4. The method of claim 3, wherein the neurodegenerative disease or disorder is Parkinson's disease.

5. The method of claim 3, wherein the neurodegenerative disease or disorder is Alzheimer's disease.

6. The method of claim 3, wherein the degenerative neurodegenerative disease or disorder is Huntington's disease or another polyglutamine disease.

7. The method of claim 3, wherein the neurodegenerative disease or disorder is amyotrophic lateral sclerosis or multiple system atrophy.

8. The method of claim 3, wherein the neurodegenerative disease or disorder is Lewy body dementia.

9. The method of claim 1, wherein the therapeutic agent is a compound that binds to PGK1 and activates PGK1 and that does not bind to the α₁-adrenergic receptor.

10. The method of claim 1, wherein the therapeutic agent is a compound that binds to PGK1 with a dissociation constant (K_{d}(PGK1)) of less than about 0.1 μM and activates PGK1 and that does not bind to the α₁-adrenergic receptor (α₁AR) or binds to the α₁AR but with a dissociation constant (K_{d}(α₁AR)) greater than about 10 μM.

11. The method of claim 10, wherein the ratio K_{d}(PGK1)/K_{d}(α₁AR) is greater than about 10.

12. The method of claim 1, wherein the therapeutic agent is a compound having the following formula or a salt or hydrate thereof: 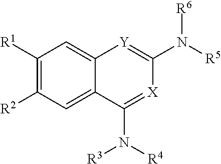
wherein:
X and Y are independently selected from CH and N, preferably at least one of X and Y is N; more preferably at least X is N; even more preferably X is N and Y is CH;
R¹ and R² are independently selected from hydrogen, alkyl, alkoxy, halo, alkylhalo, amino, cyano, and phenyl;
R³ and R⁴ are independently selected from hydrogen and alkyl;
R⁵ and R⁶ are independently selected from hydrogen, alkyl, or 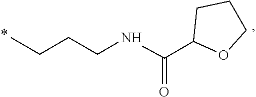
or R⁵ and R⁶ form a 5-membered or 6-membered homocycle or heterocycle (or two fused 5-membered or 6-membered homocycles or heterocycles) which is saturated or unsaturated at one or more bonds and optionally is substituted to include one or more non-hydrogen substituents, which non-hydrogen substituents optionally are selected from alkyl, halo, haloalkyl, hydroxyl, phenyl, amino, and carbonyl, and in particular R⁵ and R⁶ may form piperazinyl or a substituted piperazinyl, and optionally R⁵ and R⁶ form substituted piperazinyl having a formula 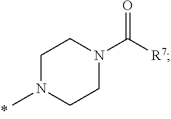
R⁷ is alkyoxy, or R⁷ is a one 3-membered ring, one 4-membered ring, one 5-membered ring, one 6-membered ring, or one 7-membered ring which ring is optionally saturated or unsaturated, or R⁷ is two fused rings which may be 5-membered rings or 6-membered rings which rings are optionally saturated or unsaturated, which one ring or two fused rings are carbocycles or heterocycles including one or more heteroatoms, which one ring or two fused rings optionally are substituted to include one or more non-hydrogen substituents, which non-hydrogen substituents optionally are selected from alkyl, halo, haloalkyl, hydroxyl, phenyl, amino, and carbonyl.

13. The method of claim 1, wherein the therapeutic agent is a compound having the following formula or a salt or hydrate thereof: 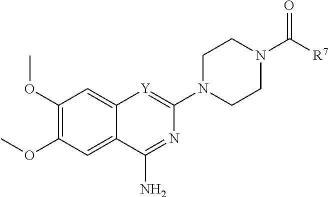
wherein:
Y is CH or N, and preferably Y is CH;
R⁷ is alkyoxy, or R⁷ is one 3-membered ring, one 4-membered ring, one 5-membered ring, one 6-membered ring, or one 7-membered ring which ring is optionally saturated or unsaturated, or R⁷ is two fused rings which may be 5-membered rings or 6-membered rings which rings are optionally saturated or unsaturated, which one ring or two fused rings are carbocycles or heterocycles including one or more heteroatoms, which one ring or two fused rings optionally are substituted to include one or more non-hydrogen substituents, which non-hydrogen substituents optionally are selected from alkyl, halo, haloalkyl, hydroxyl, phenyl, amino, and carbonyl.

14. The method of claim 1, wherein the therapeutic agent is selected from terazosin, prazosin, doxozosin, alfuzosin, trimazosin, and abanoquil or pharmaceutical salts or hydrates thereof: [chemical structure]
